(19) United States
(12) Patent Application Publication
(10) Pub. No.: US 2026/0345698 A1
(43) Pub. Date: Nov. 5, 2026

(54) METHODS FOR REDUCING ANXIETY DISORDERS OR SYMPTOMS THEREOF

(71) Applicant: BIOHM HEALTH INC., Cleveland, OH (US)

(72) Inventors: Afif Ghannoum, Moreland Hills, OH (US); Robert Fernald, Lakewood, OH (US); Michael Haritakis, Hudson, OH (US)

(21) Appl. No.: 19/108,955

(22) PCT Filed: Sep. 6, 2023
(86) PCT No.: PCT/US2023/073525

§ 371 Date: Mar. 5, 2025

Related U.S. Application Data
(60) Provisional application No. 63/404,144, filed on Sep. 6, 2022.

(51) Int. Cl. A61K 35/747; A61K 9/16; A61K 35/00; A61K 35/745; A61K 36/064; A61K 38/47; A61P 25/22

(52) U.S. Cl. CPC A61K 35/747; A61K 9/1617; A61K 9/1652; A61K 35/745; A61K 36/064; A61K 38/47; A61P 25/22; C12Y 302/01001; A61K 2035/115

(57) ABSTRACT

Methods and compositions for reducing anxiety disorders (e.g., stress) or symptoms thereof in a subject include probiotic bacterial and fungal organisms.

### CROSS-REFERENCE TO RELATED APPLICATIONS

The application claims the benefit of U.S. Provisional Patent Application No. 63/404,144 filed Sep. 6, 2022, entitled “METHODS FOR REDUCING ANXIETY DISORDERS OR SYMPTOMS THEREOF,” the contents of which are herein incorporated by reference in their entirety.

### FIELD

The present disclosure relates generally to methods for reducing anxiety disorders (for example, stress) or one or more symptoms thereof in a subject in need thereof. In particular, the present disclosure discusses the effects of probiotic supplements on reducing anxiety disorders.

### BACKGROUND

Digestive health has emerged as an important topic for many consumers and the use of microbes as medicine has been steadily gaining traction as increased understanding of the role of the microbiome in health and disease has emerged (see Zmora et al. (2019) SCI. TRANSL. MED. 11, (477)). This increased understanding of the microbiome can be attributed to improved genomics, metagenomics, and metabolomics platforms (see Id.). Probiotics, live microorganisms (bacteria and yeast) that reside primarily in the gastrointestinal tract and confer a health benefit to the host when taken in adequate amounts, are becoming a popular dietary supplement. (see Hill et al. (2014) NAT. REV. GASTROENTEROL. HEPATOL. 11(8):506-14).

The microbial specificity of each host site is dependent upon the habitat provided by the host and the commensal organisms that thrive in each habitat. The host intestinal tract (gut) represents the richest niche for microbial interactions that often feature inter-kingdom interactions such as those between bacteria (bacteriome) and fungi (mycobiome) (see Mukherjee et al. (2015) NAT. REV. GASTROENTEROL. HEPATOL. 12(2):77-87). The gut represents a diverse area within the human host, with localized areas of microbial and fungal interactions. However, the diversity represented within the host gut can be altered by exogenous and endogenous factors including diet, lifestyle (e.g., exercise, sleep, stress, etc.), generalized health, drugs and therapeutics, or other exogenous environmental factors (i.e., geographical location). Probiotics may beneficially modulate the gut microbiota in several ways including, the expansion of beneficial bacteria and yeast, increasing the mucus layer to improve the physiological barrier function within epithelial cells of the gut mucosa, and stimulating immune cells within the gut (see Butel et al. (2013) MED MAL INFECT. 44(1):1-8). Outside of the gut, probiotics may also have beneficial effects on anxiety, stress, and improving mood through the gut-brain axis (see Wallace et al. (2017) ANN GEN PSYCHIATRY 16:14. Epub 2017 Feb. 28). There are a number of different probiotic strains, and the beneficial effects may be strain specific, therefore strain blends have become popular and promising (see Id.). However, intra- as well as inter-species interactions are important regulatory factors for consideration. For example, more research is needed to examine strain specific implications and formulate a mixture of strains to provide an optimal benefit for the host at an adequate dosage to prove the effectiveness of probiotics.

### SUMMARY

The technology disclosed herein is based, in part, upon the discovery that it is possible to alleviate anxiety disorder or one or more symptoms thereof in a subject by administering to the subject a composition or a dosage form comprising both non-pathogenic bacteria and fungi, which when administered alleviates the one or more symptoms of the anxiety disorder in the subject. The present disclosure relates generally to compositions and methods for alleviating an anxiety disorder or one or more symptoms thereof in a mammal.

In one aspect, the disclosed technology provides a method of alleviating an anxiety disorder or one or more symptoms thereof in a subject in need thereof, the method comprising consumption by, or administration to, the subject of a composition comprising (i) an isolated and viable non-pathogenic fungal strain, and (ii) an isolated and viable non-pathogenic bacterial strain, thereby to alleviate the anxiety disorder or one or more symptoms thereof in the subject. In some embodiments, the disclosed technology provides a method of alleviating one or more symptoms of an anxiety disorder in a subject in need thereof, the method comprising consumption by, or administration to, the subject of a composition comprising (i) an isolated and viable non-pathogenic fungal strain, and (ii) an isolated and viable non-pathogenic bacterial strain, thereby to alleviate the one or more symptoms of an anxiety disorder in the subject. The composition comprises: (i) one or more (for example, 1, 2, 3, 4, 5 or more) isolated non-pathogenic fungal strains, and (ii) one or more (for example, 1, 2, 3, 4, 5 or more) isolated non-pathogenic bacterial strains. The alleviation of anxiety disorder or one or more symptoms thereof can be measured using any well-known method. For example, alleviation can be measured by reduction in Generalized Anxiety Disorder 7-Item (GAD-7) Scale.

In another aspect, the disclosed technology provides a composition comprising (i) an enzyme, (ii) a non-pathogenic fungal strain, and (iii) a non-pathogenic bacterial strain. For example, the composition comprises: (i) one or more (for example, 1, 2, 3, 4, 5 or more) enzymes capable, for example, of disrupting a biofilm and/or breaking down fiber in the subject, (ii) one or more (for example, 1, 2, 3, 4, 5 or more) non-pathogenic fungal strains that are viable (e.g., with or without replication), and (iii) one or more (for example, 1, 2, 3, 4, 5 or more) non-pathogenic bacterial strains that are viable (e.g., with or without replication) in the subject.

In certain embodiments, the enzyme is selected from the group consisting of amylase, cellulase, hemicellulase, lysozyme, pectinase, DNase I, Serratia peptidase or Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, and phytase. In certain embodiments, the enzyme is an amylase (for example, an amylase enzyme selected from the group consisting of Bacillus stearothermophilus amylase, Bacillus amyloliquefaciens amylase, Bacillus subtilis amylase, Bacillus licheniformi amylase, Aspergillus niger amylase, and Aspergillus oryzae amylase). In certain embodiments, the composition comprises from about 100 to about 5,000 SKB units of amylase, from about 200 to about 4,000 SKB units of amylase, from about 300 to about 2,000 SKB units of amylase, or from about 400 to about 1,000 SKB units of amylase. In certain embodiments, the composition comprises about 500 SKB units of amylase.

The composition of the present disclosure comprises at least two (for example, 2, 3, 4, 5 or more) different non-pathogenic bacterial strains. In some embodiments, the non-pathogenic bacterial strain is selected from: Lactobacillus rhamnosus, Bifidobacterium breve, Lactobacillus acidophilus, Bifidobacterium bifidum, and Lactobacillus reuteri. In some embodiments, the non-pathogenic bacterial strain is selected from: Lactobacillus rhamnosus LB 20, Bifidobacterium breve S 46, and Lactobacillus acidophilus RP 32. In some embodiments, the non-pathogenic bacterial strain comprises Lactobacillus rhamnosus LB 20, Bifidobacterium breve S 46, and Lactobacillus acidophilus RP 32.

The composition of the present disclosure comprises a non-pathogenic fungal strain. For example, Saccharomyces boulardii, Saccharomyces cerevisiae, Saccharomyceses sp HZ178, Saccharomyces bayanus, Pichia burtonii, Pichia jadinii, Pichia kudriavzevii, Pichia onychis, Pichia sp., and Picoa juniper. In some embodiments, the non-pathogenic fungal strain is Saccharomyces boulardii SB 48.

In certain embodiments, the composition is formulated as a powdered blend of one or more isolated and viable non-pathogenic bacterial strains and one or more isolated and viable non-pathogenic fungal strains. In certain embodiments, the powdered blend of one or more isolated and viable non-pathogenic bacterial strains and one or more isolated and viable non-pathogenic fungal strains is freeze dried or spray dried. In certain embodiments, the composition is freeze dried or spray dried. In certain embodiments, the freeze dried or spray dried blend of one or more isolated and viable non-pathogenic bacterial strains and one or more isolated and viable non-pathogenic fungal strains is further blended with an enzyme (for example, amylase) capable of disrupting a biofilm.

In some embodiments, the composition is disposed in a capsule. In some embodiments, the composition is formulated as a granulate, pellet, or powder. In some embodiments, the capsule comprises from about 50 mg to about 1,000 mg of the composition. In some embodiments, the capsule comprises about 500 mg to about 700 mg of the composition.

The composition of the present disclosure can further comprise an excipient (for example, a sugar, a polysaccharide (for example, a modified polysaccharide such as hydroxypropyl methylcellulose or a maltodextrin), a sugar alcohol, or an amino acid).

In certain embodiments, the composition comprises about 50 billion, about 40 billion, about 30 billion, about 20 billion, about 10 billion, about 1 billion, about 500 million, about 100 million, about 50 million, or about 10 million colony forming units of the non-pathogenic bacteria strains and non-pathogenic fungal strain(s). In certain embodiments, the composition comprises from about 10 billion to about 40 billion, from about 15 billion to about 40 billion, from about 20 billion to about 40 billion, from about 10 billion to about 30 billion, from about 15 billion to about 30 billion, from about 20 billion to about 30 billion colony forming units of the non-pathogenic bacterial strain(s). In certain embodiments, the composition comprises about 30 billion colony forming units of the non-pathogenic bacteria strains and non-pathogenic fungal strain(s). In certain embodiments, the composition comprises from about 1 billion to about 10 billion, from about 2 billion to about 8 billion, from about 3 billion to about 6 billion colony forming units of the non-pathogenic fungal strain(s). In certain embodiments, the composition comprises about 15 billion colony forming units of Bifidobacterium breve, about 10 billion colony forming units of Lactobacillus rhamnosus, about 3.5 billion colony forming units of Saccharomyces boulardii, about 1.5 billion colony forming units of Lactobacillus acidophilus, and 500 SKP of Amylase. In certain embodiments, the composition comprises an admixture (blend) of one or more isolated and viable non-pathogenic bacterial strains and one or more isolated and viable non-pathogenic fungal strains is in the form of a powder.

Other features and advantages of the disclosed technology will be apparent from the following detailed description and claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the present disclosure. In the following description, various embodiments of the present disclosure are described with reference to the following drawings, in which:

FIGS. 1A-1B are graphs showing compositional differences in gut microbiota between treatment groups at the Phyla level. Relative abundance of bacteria (16S-bacteriome) and fungal (ITS-mycobiome) composition is shown for before (Pre) and after (Post) active probiotic (PRO) and placebo (PLA) consumption.

FIGS. 2A-2B represent multi-dimensional scaling (MDS) of Bray-Curtis similarity between OUT profiles from distinct component analysis of gut bacteria (bacteriome) composition of subjects before (Pre) and after (Post) active probiotic (PRO) and placebo (PLA) consumption. FIGS. 2C-2D represent multi-dimensional scaling (MDS) of Bray-Curtis similarity between OUT profiles from distinct component analysis of fungi (mycobiome) composition of subjects before (Pre) and after (Post) active probiotic (PRO) and placebo (PLA) consumption.

FIG. 3 is a box plot showing alpha-diversity of the bacterial and fungal biomes for each sample that passed quality control between Pre and Post time points in either the active probiotic (PRO) or placebo (PLA) cohort.

FIGS. 4A-4B are plots showing statistically significant microbiota changes (p≤0.05) in Abundance (A) and Prevalence (B) in active (PRO) versus placebo (PLA) at the Post-exposure timepoint.

FIGS. 5A-5D are graphs showing results from unpaired abundance analysis in the active probiotic (PRO) (FIGS. 5A-5B) and placebo (PLA) (FIGS. 5C-5D) treatment cohorts.

FIG. 6 is a Spearman rank correlation heatmap showing correlation among clinical features and microbial abundance. Spearman rank correlation coefficients are represented by the heatmap, with red, blue, and gray indicating perfect positive, negative, and no correlation, respectively. Color of boxes indicates correlation coefficient (r) values, with darker colors indicating greater relevance (Rho).

FIG. 7 is a schematic representation of an exemplary process for making a probiotic composition described herein.

### DETAILED DESCRIPTION

The disclosed technology is based, in part, upon the discovery that it is possible to improve an anxiety disorder or one or more symptoms thereof in a subject by administering to the subject a composition or dosage form comprising both isolated and viable non-pathogenic bacteria and non-pathogenic fungi, which when administered alleviates the anxiety disorder or one or more symptoms associated thereof in the subject. Unless the context dictates otherwise, the terms composition and dosage form can be used interchangeably herein, where a dosage form is a composition and vice versa. For example, it is contemplated that the composition consumed by, or administered to, a subject can be consumed or administered as a dosage form, for example, as a unit (for example, a spoonful) of the composition. Similarly, it is understood that a dosage form can, for example, comprise a composition described herein. For example, a dosage form can comprise, for example, a composition in the form of a powder described herein, or, for example, a capsule that contains such a composition.

In one aspect, the disclosed technology provides a method of alleviating an anxiety disorder or one or more symptoms thereof in a subject in need thereof by administering to the subject a composition disclosed herein. In certain embodiments, the disclosed technology provides a method of alleviating one or more symptoms of an anxiety disorder in a subject in need thereof by administering to the subject a composition disclosed herein. In certain embodiments, the composition comprises (i) an isolated and viable non-pathogenic fungal strain, and (ii) an isolated and viable non-pathogenic bacterial strain. The composition comprises: (i) one or more (for example, 1, 2, 3, 4, 5 or more) isolated non-pathogenic fungal strains that are viable in a preselected region of the subject, and (ii) one or more (for example, 1, 2, 3, 4, 5 or more) isolated non-pathogenic bacterial strains that are viable in the region of the subject.

In some embodiments, the composition comprises (i) an enzyme, (ii) a non-pathogenic fungal strain, and (iii) a non-pathogenic bacterial strain. For example, the composition comprises: (i) one or more (for example, 1, 2, 3, 4, 5 or more) enzymes capable of disrupting a biofilm and/or breaking down fiber in a subject, (ii) one or more (for example, 1, 2, 3, 4, 5 or more) non-pathogenic fungal strains that are viable (e.g., with or without replication), and (iii) one or more (for example, 1, 2, 3, 4, 5 or more) non-pathogenic bacterial strains that are viable (e.g., with or without replication) in the subject.

In some embodiments, the composition is provided in a dosage form. The dosage form comprises a composition comprising (i) one or more (for example, 1, 2, 3, 4, 5 or more) enzymes capable of disrupting the biofilm and/or breaking down fiber in the subject, (ii) one or more (for example, 1, 2, 3, 4, 5 or more) non-pathogenic fungal strains that are viable (e.g., with or without replication) in the region of the subject, and (iii) one or more (for example, 1, 2, 3, 4, 5 or more) non-pathogenic bacterial strains that are viable (e.g., with or without replication) in the region of the subject.

The disclosed technology provides compositions or dosage forms that can alleviate an anxiety disorder or one or more symptoms thereof in a subject in need thereof. In certain embodiments, the disclosed technology provides compositions or dosage forms that can alleviate one or more symptoms of an anxiety disorder in a subject in need thereof.

In certain embodiments, the composition is formulated as a powdered blend of one or more isolated and viable non-pathogenic bacterial strains and one or more isolated and viable non-pathogenic fungal strains. In certain embodiments, the powder blend of one or more isolated and viable non-pathogenic bacterial strains and one or more isolated and viable non-pathogenic fungal strains is freeze dried or spray dried. In certain embodiments, the freeze dried or spray dried blend of one or more isolated and viable non-pathogenic bacterial strains and one or more isolated and viable non-pathogenic fungal strains is further blended with an enzyme (for example, amylase).

In certain embodiments, the composition of the disclosed technology comprises (i) one or more enzymes capable of disrupting a biofilm and/or breaking down fiber in a subject (e.g., a preselected region of the subject), (ii) one or more non-pathogenic fungal strains that are viable (e.g., with or without replication) in the subject, and (iii) one or more non-pathogenic bacterial strains that are viable (e.g., with or without replication) in the subject. The subject may be a mammal (e.g., human, a companion animal (e.g., dog, cat, or rabbit), or livestock animal (for example, cow, sheep, pig, goat, horse, donkey, and mule, buffalo, oxen, or camel)).

The composition or dosage form comprises one or more (e.g., 2, 3, 4, 5, 6, 7, 8, 9, 10, or more) different non-pathogenic bacterial strains capable of being viable or replicating (e.g., replicating in a preselected region (e.g., gastrointestinal tract, urinary tract, reproductive tract, upper respiratory tract, lower respiratory tract, biliary tract, mouth, eye, ear, or skin)) in a subject.

In certain embodiments, exemplary bacterial strains to be included in the composition or dosage form of the present disclosed technology may comprise bacterial strains of any one or more of the following bacterial species: Agrococcus jenensis, Alistipes indistinctus, Alistipes massiliensis, Alkalibacterium iburiense, Anoxybacillus kestanbolensis, Bacillus cereus, Bacillus clausii, Bacillus Coagulans, Bacteroides coprophilus, Bacteroides eggerthii, Bacteroides ovatus, Bacteroides fragilis, Bacteroides plebeius, Bacteroides uniformis, Bifidobacterium adolescentis, Bifidobacterium animalis, Bifidobacterium bifidum, Bifidobacterium breve, Bifidobacterium longum, Bifidobacterium pseudolongum, Blautia obeum, Blautia product, Candidatus azobacteroides, Candidatus portiere, Candidatus Portiera, Clostridium celatum, Clostridium hiranonis, Clostridium neonatale, Clostridium perfringens, Clostridium tyrobutyricum, Collinsella aerofaciens, Collinsella stercoris, Coprococcus eutactus, Corynebacterium stationis, Desulfosporosinus meridiei, Desulfovibrio D168, Dorea formicigenerans, Eggerthella lenta, Erwinia oleae, Faecalibacterium prausnitzii, Lactobacillus agilis, Lactobacillus reuteri, Lactobacillus ruminis, Lactobacillus salivarius, Lactobacillus zeae, Listeria weihenstephanensis, Paenibacillus mucilaginosus, Parabacteroides distasonis, Pediococcus acidilactici, Peptostreptococcus anaerobius, Prevotella copri, Prevotella melaninogenica, Prevotella stercorea, Propionibacterium acnes, Pseudoramibacter eubacterium, Roseburia faecis, Rothia dentocariosa, Rothia mucilaginosa, Ruminococcus bromii, Ruminococcus callidus, Ruminococcus flavefaciens, Ruminococcus lavefaciens, Ruminococcus gnavus, Ruminococcus torques, Salinibacillus aidingensis, Staphylococcus sciuri, Streptococcus anginosus, Streptococcus sobrinus, Tissierella soehngenia, Veillonella dispar, and Veillonella parvula.

In certain embodiments, one or more of the bacterial strains listed in TABLE 1 below are included in the composition or dosage form of the present disclosed technology.

TABLE 1

| | |
|---|---|
| Bifidobacterium adolescentis | Bacillus clausii |
| Bifidobacterium animalis | Agrococcus jenensis |
| Bifidobacterium pseudolongum | Propionibacterium acnes |
| Pseudoramibacter eubacterium | Bacteroides uniformis |
| Faecalibacterium prausnitzii | Bacteroides eggerthii |
| Dorea formicigenerans | Alistipes massiliensis |
| Blautia producta | Collinsella aerofaciens |
| Bacillus cereus | Candidatus Portiera |
| Eggerthella lenta | Coprococcus eutactus |
| Ruminococcus bromii | Collinsellas tercoris |
| Bifidobacterium longum | Prevotella melaninogenica |
| Veillonella dispar | Clostridium tyrobutyricum |
| Parabacteroides distasonis | Ruminococcus gnavus |
| Ruminococcus callidus | Anoxybacillus kestanbolensis |
| Salinibacillus aidingensis | Ruminococcus flavefaciens |
| Staphylococcus sciuri | Lactobacillus rhamnosus |
| Desulfosporosinus meridiei | Bifidobacterium breve |
| Lactobacillus zeae | Lactobacillus acidophilus |
| Roseburia faecis | Bifidobacterium bifidum |
| Clostridium perfringens | Lactobacillus reuteri |
| Veillonella parvula | |

In certain embodiments, one or more of the bacterial strains listed in TABLE 2 below are included in the composition or dosage form of the present disclosed technology.

TABLE 2

Lactobacillus rhamnosus
Bifidobacterium breve
Lactobacillus acidophilus
Bifidobacterium bifidum
Lactobacillus reuteri

In certain embodiments, the composition or dosage form comprises one, two or three different bacterial strains (e.g., strains listed in TABLE 1 or 2) capable of replicating in the region of the mammal.

In certain embodiments, the non-pathogenic bacterial strains included in the composition or dosage forms of the present disclosed technology may comprise a combination of any two of Lactobacillus rhamnosus LB 20, Bifidobacterium breve S 46, and Lactobacillus acidophilus RP 32. In certain embodiments, the non-pathogenic bacterial strains included in the composition or dosage form may comprise a combination of Lactobacillus rhamnosus LB 20 and Bifidobacterium breve S 46. In certain embodiments, the non-pathogenic bacterial strains included in the composition or dosage form may comprise a combination of Lactobacillus rhamnosus LB 20 and Lactobacillus acidophilus RP 32. In certain embodiments, the non-pathogenic bacterial strains included in the composition or dosage form may comprise a combination of Bifidobacterium breve S 46 and Lactobacillus acidophilus RP 32. In certain embodiments, the non-pathogenic bacterial strains included in the composition or dosage form of the present disclosed technology may comprise a combination of Lactobacillus rhamnosus LB 20, Bifidobacterium breve S 46, and Lactobacillus acidophilus RP 32.

In certain embodiments, the composition or dosage form of the technology comprises from about 10 billion to about 40 billion, e.g., from about 15 billion to about 40 billion, from about 20 billion to about 40 billion, from about 10 billion to about 30 billion, from about 15 billion to about 30 billion, from about 20 billion to about 30 billion colony forming units of the non-pathogenic bacterial strain(s).

The composition or dosage form also comprises one or more (e.g., 2, 3, 4, 5, 6, 7, 8, 9, 10, or more) different non-pathogenic fungal strains capable of replicating (e.g., in a preselected region (e.g., gastrointestinal tract, urinary tract, reproductive tract, upper respiratory tract, lower respiratory tract, biliary tract, mouth, eye, ear, or skin)) in a subject.

In certain embodiments, exemplary fungal strains to be included in the composition or dosage form of the present technology may comprise any one or more of the following fungal species: Albatrellus syringae, Alternaria alli, Alternaria daturicola, Ambispora granatensis, Amyloathelia crassiuscula, Amylomyces rouxii, species of Ascomycota, Ascosphaera apis, Aspergillus flavus, Aspergillus fumigatus, Aspergillus oryzae, Aspergillus sp, Aspergillus terreus, Aspergillus versicolor, Bettsia alvei, Botryosphaeria mamane, Botryotinia fuckeliana, Candida arabinofermentans, Candida ernobii, Candida ethanolica, Candida glabrata, Candida humilis, Candida intermedia, Candida parapsilosis, Candida piceae, Candida quercitrusa, Candida tartarivorans, Candida temnochilae, Candida zemplinina, Candida zeylanoides, Chamonixia caespitosa, Cladonia polystomata, Cladosporium cladosporioides, Cladosporium halotolerans, Cladosporium sp JP67, Cladosporium sphaerospermum, Clavicorona taxophila, Clavispora lusitaniae, Craterellus sp, Dactylellina phymatopaga, Debaryomyces hansenii, Debaryomyces marasmus, Debaryomyces sp, Debaryomyces subglobosus, Dipodascus australiensis, Ectomycorrhizal, Emericella nidulans, Epulorhiza sp, Eremascus fertilis, Eremothecium gossypii, Eupenicillium cinnamopurpureum, Eurotium amstelodami, Eurotium cristatum, Exophiala dermatitidis, Filobasidiella neoformans, Fonsecaea monophora, Fusarium solani, Fusarium dimerum, Fusarium oxysporum, Fusarium sp, Galactomyces geotrichum, Galactomyces sp, Geomyces sp, Geotrichum cucujoidarum, Geotrichum sp, Glomus mosseae, Hanseniaspora sp, Helicobasidium longisporum, Helicostylum pulchrum, Hyphodontia flavipora, Hypochnicium cystidiatum, Inocybe sp, Kazachstania africana, Kazachstania unispora, Kluyveromyces lactis, Kluyveromyces yarrowii, Kodamaea ohmeri, Leptosphaeria biglobosa, Leptosphaerulina chartarum, Leucoagaricus sp, Lewia infectoria, Lichtheimia ramose, Macrophomina phaseolina, Monacrosporium coelobrochum, Mucor circinelloides, Mucor flavus, Mucor fuscus, Mucor sp, Myrothecium sp, Myxozyma melibiosi, Neocallimastix frontalis, Neocallimastix sp, Omphalotus nidiformis, Ophiocordyceps filiformis, Ophiocordyceps sinensis, Penicillium carneum, Penicillium chrysogenum, Penicillium concentricum, Penicillium crustosum, Penicillium digitatum, Penicillium granulatum, Penicillium griseofulvum, Penicillium griseoroseum, Penicillium polonicum, Penicillium psychrosexualis, Penicillium pulvillorum, Penicillium roqueforti, Penicillium sclerotigenum, Penicillium sp, Penicillium spinulosum, Penicillium verrucosum, Pezizomycetes sp genotype 323, Phaeophyscia exornatula, Phialocephala lagerbergii, Phlebia radiata, Phlebia sp, Phomopsis sp, Physcia stellaris, Physoderma maydis, Pichia burtonii, Pichia jadinii, Pichia kudriavzevii, Pichia onychis, Pichia sp, Picoa juniperi, Pilaira cesatii, Pilaira sp, Pirella circinans, Preussia sp, Reddellomyces donkii, Rhizomucor pusillus, Rhizopus oryzae, Rhodosporidium kratochvilovae, Rhodosporidium sp, Rhodotorula sp, Rinodina milvina, Saccharomyces bayanus, Saccharomyces bulderi, Saccharomyces cerevisiae, Saccharomyces mikatae, Saccharomyces sp, Saccharomyces sp, Scleroderma sp, Sclerotinia sp, Scutellospora nodose, Sebacinales sp, Sporopachydermia sp, Stenocarpella maydis, Thamnidium elegans, Torulaspora delbrueckii, Trichosporon chiarellii, Tuber oligospermum, Umbelopsis isabellina, Wallemia sebi, Wallemia sp, and Zygoascus meyerae.

In certain embodiments, the composition or dosage form may comprise a non-pathogenic fungal strain selected from Saccharomyces boulardii, Saccharomyces cerevisiae, Saccharomyces sp HZ178, Saccharomyces bayanus, strains belonging to the Pichia genus (for example, including but limited to Pichia burtonii, Pichia jadinii, Pichia kudriavzevii, Pichia onychis, and Pichia juniper).

In certain embodiments, the composition or dosage form comprises a composition comprising a non-pathogenic fungal strain Saccharomyces boulardii SB 48. In certain embodiments, the composition or dosage form of the presently disclosed technology comprises a non-pathogenic fungal strain Saccharomyces boulardii SB 48 and one or more of the bacterial strains listed in TABLE 1 or TABLE 2.

In certain embodiments, the composition or dosage form comprises from about 1 billion to about 10 billion, e.g., from about 2 billion to about 8 billion, from about 3 billion to about 6 million colony forming units of the non-pathogenic fungal strain(s).

In certain embodiments, the composition or dosage form comprises a non-pathogenic fungal strain Saccharomyces boulardii SB 48 and non-pathogenic bacterial strains of one or more of Lactobacillus rhamnosus LB 20, Bifidobacterium breve S 46, and Lactobacillus acidophilus RP 32. In certain embodiments, the composition or dosage form comprises a non-pathogenic fungal strain Saccharomyces boulardii SB 48 and non-pathogenic bacterial strains Lactobacillus rhamnosus LB 20 and Bifidobacterium breve S 46. In certain embodiments, the composition or dosage form comprises a non-pathogenic fungal strain Saccharomyces boulardii SB 48 and non-pathogenic bacterial strains Lactobacillus rhamnosus LB 20 and Lactobacillus acidophilus RP 32. In certain embodiments, the composition or dosage form comprises a non-pathogenic fungal strain Saccharomyces boulardii SB 48 and non-pathogenic bacterial strains Bifidobacterium breve S 46 and Lactobacillus acidophilus RP 32. In certain embodiments, the composition or dosage form comprises a non-pathogenic fungal strain Saccharomyces boulardii SB 48 and the non-pathogenic bacterial strains Lactobacillus rhamnosus LB 20, Bifidobacterium breve S 46, and Lactobacillus acidophilus RP 32.

In certain embodiments, the composition or dosage form comprises from about 15 billion colony forming units of Bifidobacterium breve, about 10 billion colony forming units of Lactobacillus rhamnosus, about 3.5 billion colony forming units of Saccharomyces boulardii, and about 1.5 billion colony forming units of Lactobacillus acidophilus.

In addition to non-pathogenic bacteria and fungi, the composition or dosage form further comprises an enzyme capable of disrupting the biofilm and/or breaking down fiber. For example, the enzyme preferably digests or otherwise disrupts/breaks down the EPS matrix of the biofilm.

The enzyme can be selected from amylase, cellulase, hemicellulase, lysozyme, pectinase, DNase I, Serratia peptidase, Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, phytase, zymolase and nuclease. The enzyme may be chosen depending upon the type of biofilm and the microorganisms disposed therein. For example, an amylase enzyme may be used to degrade or otherwise disrupt carbohydrate components of the biofilm, and a nuclease such a DNase I may be used for digest or otherwise disrupt DNA in the biofilm.

In certain embodiments, the composition or dosage form comprises two or more (e.g., 2, 3, 4, 5, or more) different enzymes selected from amylase, cellulase, hemicellulase, lysozyme, pectinase, DNase I, Serratia peptidase, Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, phytase, zymolase and nuclease.

In certain embodiments, a composition or dosage form described hereinabove comprises an amylase selected from Bacillus stearothermophilus amylase, Bacillus amyloliquefaciens amylase, Bacillus subtilis amylase, Bacillus licheniformi amylase, Aspergillus niger amylase, and Aspergillus oryzae amylase.

In certain embodiments, the composition or dosage form comprises an amylase, for example, from about 100 to about 5,000 SKB units of amylase, from about 200 to about 4,000 SKB units of amylase, from about 300 to about 2,000 SKB units of amylase or from about 400 to about 1,000 SKB units of amylase. An SKB or Sandstedt, Kneen, and Blish unit refers to the amount of amylase to catalyze 1 μmole substrate per minute. In certain embodiments, composition or dosage form comprises a cellulose, for example, and comprises from about 100 to about 300 CU (Cellulase unit) units per unit composition or dosage form, for example, about 200 CU. In certain embodiments, composition or dosage form comprises a hemicellulose/pectinase complex, and comprises from about 60 to about 100 HSU (Hemicellulose Specific Units) units per unit composition or dosage form, for example, about 80 HSU. In certain embodiments, the composition or dosage form comprises a β-gluconase, and comprises from about 6 to about 10 BGU (Beta Glucanase Unit), units per unit composition or dosage form, for example, about 8 BGU. In certain embodiments, the composition or dosage form comprises an acid protease, and comprises from about 15 to about 25 SAP (Shrimp Alkaline Phosphatase) units per unit composition or dosage form, for example, about 20 SAP units. In certain embodiments, the composition or dosage form comprises alkaline protease, and comprises from about 15 to about 25 HUT (hemoglobin unit Tyrosine base) units per unit composition or dosage form, for example, about 20 HUT units.

In certain embodiments, total amount of cellulase administered, for example, by the administration of one or more units of the composition or dosage form, may range from about 1 to about 10,000 CU, the total amount of hemicellulase/pectinase complex administered, for example, by the administration of one or more units of the composition or dosage form, may range from about 1 to about 8,000 HSU, the total amount of β-gluconase, for example, by the administration of one or more dosage units, may range from about 1 to about 1000 BGU, the total amount of acid protease, for example, by the administration of one or more dosage units, may range from about 1 to about 10,000 SAP, and the total amount of alkaline protease, for example, by the administration of one or more dosage units, may range from about 1 to about 40,000 HUT.

In certain embodiments, the composition or dosage form comprises about 500 SKB units of amylase, selected from α-Amylase, an endo-hydrolase that catalyzes the hydrolysis of internal α-1, 4-glycosidic linkages in starch to yield products like glucose and maltose, β-Amylase, an exo-hydrolase enzyme that hydrolyses α-1, 4-glucan linkages to yield successive maltose units, and γ-Amylase, which cleaves α-1, 6-glycosidic linkages, in addition to cleaving the last α-1, 4-glycosidic linkages to yield glucose, or a combination thereof.

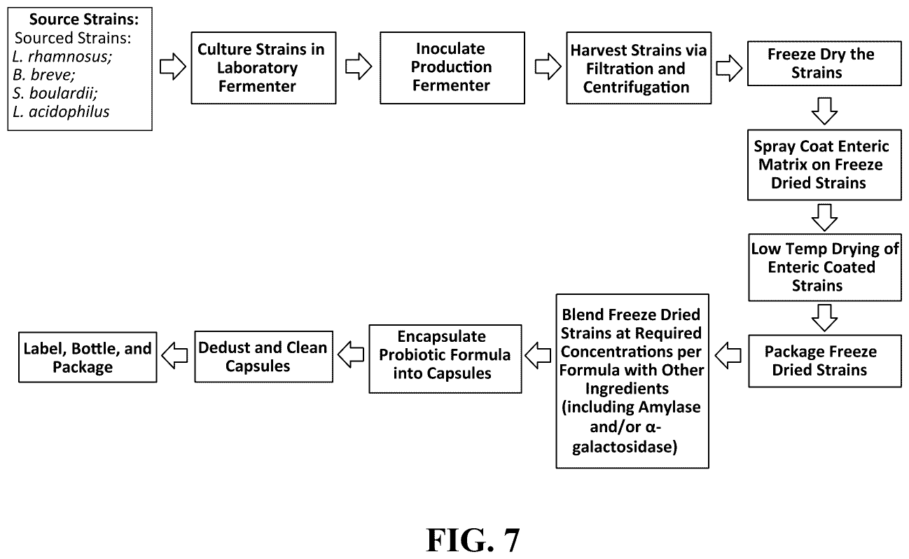

In certain embodiments, a composition of the present technology may be manufactured according to the following process: The non-pathogenic bacterial strains (e.g., Lactobacillus casei, Bifidobacterium longum, and Bifidobacterium breve) are each cultured separately in a small scale fermenter. For each strain, a culture sample is then used to inoculate a corresponding large scale production fermenter. The cultured bacterial strains are harvested via filtration and/or centrifugation, and can then be dried, for example, freeze dried or spray-dried. The dried organisms can then be coated, for example, spray coated with a polymer such as hydroxylpropylmethyl cellulose (HPMC). The coated strains, for example, HPMC-coated strains, can then be subjected to further drying, such as, low temperature drying (e.g., room temperature). The spray coating of freeze-dried bacterial strains is an optional step. The resulting strains (e.g., coated or non-coated) are then blended together at the appropriate concentrations for the specific formulation being manufactured. In order to ensure the active ingredients (e.g., bacterial strains) are at the appropriate concentrations, a manufacturing excipient can also be added. Manufacturing excipients (e.g., a sugar, a polysaccharide (e.g., a modified polysaccharide), a sugar alcohol, or an amino acid) are well-known in the art. Exemplary manufacturing excipients that can be used are leucine, maltodextrin, mannitol and any combination thereof. At this blending stage, if desired, other ingredients specific to the formulation (e.g., dried amylase or dried α-galactosidase) can be added. If desired, the blended composition can then be encapsulated, which can then be packaged into an appropriately labeled container. An exemplary manufacturing process is depicted in FIG. 7.

In certain embodiments, the composition or dosage form comprises a powder (e.g., a coated powder), comprising about 10 million-about 50 billion colony forming units (CFUs) of isolated non-pathogenic fungal strain(s) and isolated non-pathogenic bacterial strain(s). In certain embodiments, the composition or dosage form of the present technology comprises about 10 million-about 40 billion, about 10 million-about 30 billion, about 10 million-about 20 billion, about 10 million-about 10 billion, about 10 million-about 5 billion, about 10 million-about 1 billion, about 10 million-about 500 million, about 10 million-about 100 million, about 10 million-about 50 million CFUs of non-pathogenic fungal strain(s) and non-pathogenic bacterial strain(s). In certain embodiments, the composition or dosage form of the present technology comprises about 20 million-50 billion, about 50 million-50 billion, about 100 million-about 50 billion, about 200 million-about 50 billion, about 500 million-about 50 billion, about 1 billion-about 50 billion, about 5 billion-about 50 billion, about 10 billion-about 50 billion, about 20 billion-about 50 billion, about 30 billion-about 50 billion, or about 40 billion-about 50 billion CFUs of non-pathogenic fungal strain(s) and non-pathogenic bacterial strain(s). In certain embodiments, the composition or dosage form may further comprise an enzyme (e.g., amylase, cellulase, hemicellulase, lysozyme, pectinase, DNase I, Serratia peptidase, Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, phytase, zymolase and nuclease).

In certain embodiments, the composition or dosage form comprises about 50 billion, about 40 billion, about 30 billion, about 20 billion, about 10 billion, about 1 billion, about 500 million, about 100 million, about 50 million, or about 10 million CFUs of non-pathogenic fungal strain(s) and non-pathogenic bacterial strain(s). In certain embodiments, one capsule comprises about 30 billion colony forming units of non-pathogenic fungal strain(s) and non-pathogenic bacterial strain(s) and an enzyme. In certain embodiments, one capsule comprises about 15 billion colony forming units of Bifidobacterium breve, about 1.5 billion colony forming units of Lactobacillus acidophilus, about 10 billion colony forming units of Lactobacillus rhamnosus, about 3.5 billion Saccharomyces boulardii, and about 500 SKB units of amylase. In certain embodiments, a low dose of 10 million CFU is effective for use as a probiotic or a composition.

In certain embodiments, the composition or dosage form may be consumed orally by the subject. In certain embodiments, the composition is consumed as a powder or product containing the powder. In other embodiments, the composition can be in the form of an oral dosage form, for example, where the composition in included, for example, within a capsule, cachet, pill, tablet, lozenge, powder, granule, or as a solution or a suspension in an aqueous or non-aqueous liquid, or as an oil-in-water or water-in-oil liquid emulsion, or as an elixir or syrup, or as pastilles, each containing the requisite number of colony forming units of the non-pathogenic bacteria and non-pathogenic fungus, and optionally the appropriate amount of enzyme.

In certain embodiments, the composition is disposed in a capsule or in a tablet. In certain embodiments, the composition is formulated as a tablet. In certain embodiments, the capsule is a vegetable cellulose capsule. In certain embodiments, the dosage form, for example, capsule or tablet, is coated with a coating, for example, a non-functional aesthetic coating or a functional coating, for example, a controlled release coating. The capsule or tablet may be formulated so as to provide slow or controlled release of the ingredients disposed therein.

In certain embodiments, a dosage form contains a composition comprising (i) an enzyme capable of disrupting the biofilm, (ii) a non-pathogenic fungal strain capable of replicating in the region of the subject, and (iii) a non-pathogenic bacterial strain capable of replicating in the region of the subject that is formulated as a powder and encapsulated in a capsule.

In one exemplary probiotic, a capsule comprises a probiotic blend containing the ingredients set forth in TABLE 3:

TABLE 3
Probiotic Blend (Size 1 capsule)

| | | |
|---|---|---|
| Ingredient | CFU/cap | SKB/cap |

| | | |
|---|---|---|
| Lactobacillus rhamnosus | 10 | billion |
| Bifidobacterium breve | 15 | billion |
| Saccharomyces boulardii | 3.5 | billion |
| Lactobacillus acidophilus | 1.5 | billion |

| | |
|---|---|
| Amylase | 500 |

| | | | |
|---|---|---|---|
| Total | 30 | billion | 500 |

In certain embodiment, the composition or dosage form comprises additives such as calcium carbonate, xylitol, cetyl alcohol, citric acid, natural flavor, monk fruit. In certain embodiments, the composition comprises additives such as cascara sagrada bark, psyllium husk, senna leaf, Flaxseed, aloe vera leaf, licorice root, medium chain triglyceride (MCT) oil. In certain embodiments, the composition or dosage form comprises additives such as a dietary fiber, e.g., inulin (fructo oligosaccharides FOS) and apple pectin. In certain embodiments, the composition or dosage form comprises additives such as a blend of spirulina, barley grass, alfalfa leaf, wheat grass, chloerlla, dulse, spinach leaf, broccoli, parsley leaf, kale leaf, echinacea angustifolia root, licorice root, milk thistle seed, Siberian eleuthero root, beet root, rose hips, acai (fruit), green tea leaf, raspberry leaf, blueberry (fruit), goji berry, bilberry (fruit), ashwagandha root, rhodiola root, reishi mushroom, maca root, bee pollen, nettle leaf, ginko biloba (leaf extract), royal jelly (3× concentrate), grape seed. In certain embodiments, the composition further comprises a vitamin, such as, vitamin C.

The size of the dose delivered or consumed, will depend upon, among other things, the size and age of the subject, the indication or condition to be treated, and the mode of delivery of the composition or dosage form. In certain embodiments, for example, oral composition or dosage forms may comprise from about 1 mg to about 1,000 mg or 50 mg to about 1,000 mg of the active ingredients (for example, the non-pathogenic bacteria, the non-pathogenic fungus, and/or the enzyme). In certain embodiments, the composition or dosage form may comprise from about 1 mg to about 900 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 1 mg to about 500 mg, about 10 mg to about 1000 mg, about 20 mg to about 1000 mg, about 30 mg to about 1000 mg, about 40 mg to about 1000 mg, about 50 mg to about 1000 mg, about 100 mg to about 1000 mg, about 200 mg to about 1000 mg, about 300 mg to about 1000 mg, about 400 mg to about 1000 mg, about 500 mg to about 1000 mg, about 600 mg to about 1000 mg, about 700 mg to about 1000 mg, about 800 mg to about 1000 mg, or about 900 mg to about 1000 mg of the active ingredients (for example, the non-pathogenic bacteria, the non-pathogenic fungus, and/or the enzyme).

In certain embodiments, the composition or dosage form may comprise from about 100 mg to about 200 mg, from about 200 mg to about 300 mg, from about 300 mg to about 400 mg, from about 400 mg to about 500 mg, from about 500 mg to about 600 mg, from about 600 mg to about 700 mg, from about 700 mg to about 800 mg, from about 800 to about 900 mg, or from about 900 to about 1000 mg of the active ingredients. In certain embodiments, the composition or dosage form may comprise about 575 mg of the active ingredients.

In certain embodiments, the composition or dosage form comprises about 50, 40, 30, or 20 billion colony forming units of the non-pathogenic bacteria strains and non-pathogenic fungal strain(s). In certain embodiments, the composition or dosage form comprises about 30 billion colony forming units of the non-pathogenic bacteria strains and non-pathogenic fungal strain(s).

In certain embodiments, the composition or dosage form of the technology comprises from about 10 billion to about 40 billion, e.g., from about 15 billion to about 40 billion, from about 20 billion to about 40 billion, from about 10 billion to about 30 billion, from about 15 billion to about 30 billion, from about 20 billion to about 30 billion colony forming units of the non-pathogenic bacterial strain(s). Alternatively, or in addition, the composition or dosage form of the technology comprises from about 1 billion to about 10 billion, e.g., from about 2 billion to about 8 billion, from about 3 billion to about 6 million colony forming units of the non-pathogenic fungal strain(s).

In certain embodiments, the composition or dosage form comprises about 15 billion colony forming units of Bifidobacterium breve, about 10 billion colony forming units of Lactobacillus rhamnosus, about 3.5 billion colony forming units of Saccharomyces boulardii, and about 1.5 billion colony forming units of Lactobacillus acidophilus.

The composition or dosage forms (either one or multiple units (for example 2, 3, 4, or 5 units, for example, capsules or tablets) may be administered once, twice or three times a day during the treatment period, for example, until the biofilm of interest has been disrupted, prevented from forming, and/or the subject's normal microbiome has been restored which could take, for example, one week, two weeks, one month, two months, or three months and one year. In certain embodiments, one capsule, that comprises about 30 billion colony forming units of non-pathogenic fungal strain(s) and non-pathogenic bacterial strain(s) and an enzyme, is administered once, twice, or three times a day during the treatment period, for example, until the subject's normal microbiome has been restored which could take, for example, one week, two weeks, one month, two months, or three months and one year. In certain embodiments, one capsule, that comprises about 30 billion colony forming units of non-pathogenic fungal strain(s) and non-pathogenic bacterial strain(s) and an enzyme is administered once per day. In certain embodiments, the capsule administered once per day comprises about 15 billion colony forming units of Bifidobacterium breve, about 1.5 billion colony forming units of Lactobacillus acidophilus, about 10 billion colony forming units of Lactobacillus rhamnosus, about 3.5 billion Saccharomyces boulardii, and about 500 SKB units of amylase.

The compositions or dosage form of the present disclosure can be used to alleviate an anxiety disorder or one or more symptoms thereof in a subject.

The method comprises administering to the subject one or multiple units (for example, capsules or tablets) of compositions or dosage forms described herein, thereby to alleviate the anxiety disorder or one or more symptoms associated with it. The subject may be a mammal (e.g., human, a companion animal (e.g., dog, cat, or rabbit), or livestock animal (for example, cow, sheep, pig, goat, horse, donkey, and mule, buffalo, oxen, or camel)).

The term “anxiety disorder” refers to a state of apprehension, uncertainty, and/or fear resulting from the anticipation of an event and/or situation. Non-limiting examples of anxiety disorders include acute stress disorder, anxiety due to a medical condition, generalized anxiety disorder, panic disorder, panic attack, a phobia, post-traumatic stress disorder, obsessive-compulsive disorder, separation anxiety disorder, social anxiety disorder, substance-induced anxiety disorder, or selective mutism.

In some embodiments, at least one sign or symptom of an anxiety disorder is improved following the administration of the composition disclosed herein. In some embodiments, a sign or symptom of an anxiety disorder is measured according to a diary assessment, an assessment by a clinician or caregiver, or a clinical scale. In some embodiments, administration of the composition disclosed herein causes a demonstrated improvement in one or more of the following: Generalized Anxiety Disorder 7-Item Scale, State-Trait Anxiety Inventory (STAI), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale—Interview for DSM-5 (PSS-I-5), Structured Interview for PTSD (SI-PTSD), Davidson Trauma Scale (DTS), Impact of Event Scale—Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI-R), Quality of Life (QoL), The Clinical Global Improvement (CGI) scale, The Yale-Brown Obsessive-Compulsive Scale (Y-BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y-BOCS-SR), The Obsessive-Compulsive Inventory—Revised (OCI-R), and the Dimensional Obsessive-Compulsive Scale (DOCS), or a combination thereof.

An assessment using GAD-7 scale assesses the frequency of 7 items (“Feeling nervous, anxious, or on edge,” “Not being able to stop or control worrying,” “Worrying too much about different things,” “Trouble relaxing,” “Being so restless that it's hard to sit still,” “Becoming easily annoyed or irritable,” and “Feeling afraid as if something awful might happen”). A higher score on the GAD-7 indicates a greater degree of anxiety.

As used herein, the terms “treat,” “treating,” or “treatment,” and other grammatical equivalents as used herein, include alleviating, abating, ameliorating, or preventing a disease, condition or symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis.

As used herein, the terms, “subject,” “patient,” “subject in need thereof,” and “patient in need thereof” are used interchangeably herein, and refer to a living organism, including animals and humans, suffering from or prone to a disease or condition that can be treated by the methods and compositions provided herein. The subject can be a human or non-human animal.

The following definitions are included for the purpose of understanding the present subject matter and for constructing the appended patent claims.

Throughout the description, where compositions are described as having, including, or comprising specific components, or where processes and methods are described as having, including, or comprising specific steps, it is contemplated that, additionally, there are compositions of the present technology that consist essentially of, or consist of, the recited components, and that there are processes and methods according to the present technology that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present technology, whether explicit or implicit herein. For example, where reference is made to a particular composition, that composition can be used in the various embodiments of such compositions of the present technology and/or in methods of the present technology, unless otherwise understood from the context. In other words, within this application, embodiments have been described and depicted in a way that enables a clear and concise application to be written and drawn, but it is intended and will be appreciated that embodiments may be variously combined or separated without parting from the present teachings. For example, it will be appreciated that all features described and depicted herein can be applicable to all aspects of the technology described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The term “consisting essentially” as used herein means the specified materials or steps and those that do not materially affect the basic and novel characteristics of the material or method. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary. The use of the term “includes,” “has,” or “contains,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present technology also includes the specific quantitative value itself, unless specifically stated otherwise. As used herein, the term “about” refers to a ±10% variation from the nominal value unless otherwise indicated or inferred.

Where a molecular weight is provided for a polymer or oligomer the molecular weight should be understood to be a number average molecular weight, unless otherwise stated or understood from the context. As a general matter, compositions specifying a percentage are by weight unless otherwise specified.

At various places in the present specification, components, or features thereof are disclosed in groups or in ranges. It is specifically intended that the description include each and every individual sub-combination of the members of such groups and ranges. By way of other examples, an integer in the range of 1 to 5 is intended to individually disclose 1, 2, 3, 4, and 5.

Practice of the invention will be more fully understood from the foregoing examples, which are presented herein for illustrative purposes only, and should not be construed as limiting the invention in any way.

### EXAMPLES

The following Examples are merely illustrative and are not intended to limit the scope or content of the invention in any way.

### Example 1

Example 1 is a—a clinical study testing the effects of administration of a probiotic composition on reducing anxiety. This clinical study was a randomized, placebo-controlled, double blind study with 52 subjects. The study consisted of three visits. During the initial screening visit, each subject's medical history and routine blood work (complete blood count (CBC), comprehensive metabolic panel (CMP), and lipid panel) were collected, and a 24-hour dietary recall was recorded. At the next two visits, body weight was measured, followed by baseline measurements of vitals (blood pressure and heart rate), visual analog scales (VAS) for flatulence, bloating, abdominal discomfort, stool consistency/regularity and constipation, and questionnaires that assess the subject's overall well-being (e.g., SF-36), physical activity (e.g., Framingham), and general anxiety (i.e., GAD-7). On the third study visit routine blood work (CBC, CMP, and Lipid Panel) was performed. Subjects were given at-home stool kits for microbiome analysis. A summary of the research design is provided in TABLE 4.

TABLE 4

| | | Visit 2 | Visit 3 |
|---|---|---|---|
| Procedure | Screening | (Week 0) | (Week 6) |
| Informed Consent | X | | |
| Inclusion/Exclusion Criteria | X | | |
| Medical History | X | | |
| Physical | X | | |
| Anxiety (ASQ/GAD-7) Questionnaire | | X | X |
| Diet Records/Analysis/Background | X | X | X |
| Diet* | | | |
| Protocol Compliance every week | | X | X |
| Dispense Test Product | | X | |
| Adverse Events Monitoring | | X | X |

*3-day diet records conducted/collected at screening visit and during week 6.

After completion of visit 2 (baseline testing visit), subjects were randomized in a double-blind, placebo-controlled fashion to ingest a daily dose of a 575 mg of 30 billion colony forming units (CFU) probiotic blend consisting of Bifidobacterium breve 19bx, Lactobacillus acidophilus 16axg, Lactobacillus rhamnosus 18fx, and Saccharomyces boulardii 16mxg, and 500 SKB of alpha amylase (“PRO”), or a placebo consisting of rice oligodextrin (“PLA”) for 6 weeks. The complete composition of PRO is provided in TABLE 5. Prior to all study visits, subjects were asked to replicate their previous dietary intake for the 24 hours prior to their visit, refrain from caffeine for 24 hours, fast for 10 hours, and abstain from exercise for 24 hours. Comprehensive side effect profile/adverse event monitoring took place throughout the study duration. The study was conducted following international council for harmonization E6 good clinical practice (ICH-GCP) guidelines to ensure subject safety and scientific integrity of the data.

TABLE 5
Composition of Probiotic Blend (PRO)

| | |
|---|---|
| Organism | Strain |

Probiotic Organisms

| | |
|---|---|
| Lactobacillus rhamnosus | 18fx |
| Lactobacillus acidophilus | 16axg |
| Saccharomyces boulardii | 16mxg |
| Bifidobacterium breve | 19bx |

Other Ingredients

| | |
|---|---|
| Amylase | Alpha-amylase-Dextrinizing Units |
| | (500 SKB) |
| Rice Maltodextrin | |
| Vegetable Cellulose (capsule) | |
| Magnesium Stearate | |
| Amount Per Serving | |
| 575 mg (30 Billion CFU) | |

52 (14 men and 38 women) subjects were randomized into the study protocol. 27 subjects were assigned to PLA group and 25 were assigned to the PRO group. TABLE 6 presents the baseline demographics characteristics of the study cohort, showing that the two cohorts were well matched with no differences between the two groups based on demographic information.

TABLE 6

| | Total | A (Active, | B (Placebo), |
|---|---|---|---|
| | (N = 52) | N = 25) | N = 27) |
| Age (years) | 44.4 ± 8.9 | 44.0 ± 10.0 | 44.7 ± 7.9 |
| Men | 14 (26.9%) | 8 (32%) | 6 (22.2%) |
| Women | 38 (73.1%) | 17 (68%) | 21 (77.8%) |
| Height (cm) | 170.3 ± 11.5 | 171.4 ± 10.3 | 169.2 ± 12.7 |
| Weight (kg) | 82.0 ± 18.4 | 85.5 ± 18.6 | 78.7 ± 18.0 |
| Body Mass Index (kg/m²) | 28.1 ± 4.6 | 28.9 ± 4.6 | 27.4 ± 4.5 |
| Systolic Blood Pressure (mm | 123.4 ± 11.3 | 124.6 ± 10.1 | 122.3 ± 12.3 |
| Hg) | | | |
| Diastolic Blood Pressure (mm | 79.3 ± 7.0 | 79.6 ± 7.5 | 79.0 ± 6.7 |
| Hg) | | | |
| Resting Heart Rate (bpm) | 72.5 ± 10.0 | 79.0 ± 6.7 | 70.2 ± 9.4 |

Data shown as Mean ± SD. PRO = Probiotic group; PLA = Placebo group.

All subjects provided IRB-approved informed consent prior to participating in the study. Upon review of health/medical history documents and a physical exam, all study subjects were free from chronic health issues. Inclusion criteria were established so that all subjects were required to be between 30-60 years old, score ≥12 on the GSRS, have a minimum body mass of 120 pounds (54.5 kg), and body mass index (BMI) between 20.0-34.99 kg/m². Subjects were also required to be normotensive (<140/<90 mm Hg) with a normal resting heart rate (<90 beats/min). Female subjects were not eligible if they were determined to be pregnant, nursing, or trying to become pregnant. Exclusion criteria included: any history of unstable or new-onset cardiovascular or cardiorespiratory disease; any history of stroke, diabetes, or other endocrine disorder; use of any nutritional supplement known to alter the gut microbiome/microflora, probiotic supplements, use of prebiotic supplements in the previous 4 weeks and for the duration of the study; use of any antibiotics, antifungals, antivirals, or antiparasitic within 8 weeks of the start of the study or throughout the study; any changes in diet within 4 weeks of study start date or throughout study duration; if the subject was unwilling to abstain from gut altering supplements for the duration of the study; any history of malignancy in the previous five years except for non-melanoma skin cancer; any history of prior gastrointestinal bypass surgery; any history of any known gastrointestinal or metabolic diseases that might impact nutrient absorption or metabolism; any history of any chronic inflammatory condition or disease; and any history of known sensitivity to any ingredient in the test formulations as listed in the certificates of analysis.

Subjects were excluded if they were currently participating in another research study with an investigational product or had participated in another research study in the past 30 days, or if they had any other diseases or conditions that, in the opinion of the medical staff, could confound the primary endpoints or place the subject at increased risk of harm if they were to participate.

Standing height was determined using a wall-mounted stadiometer Height, Body Mass, Heart Rate, Blood Pressure—with each study subject in socks or bare feet with heels together. Body mass was measured using a Seca 767™ Medical Scale. Resting heart rate and blood pressure was measured using an automated blood pressure cuff (Omron HEM-780) after subjects had remained seated for a minimum of five minutes.

Questionnaires: The Framingham physical activity questionnaire was administered before and after 6 weeks of supplementation to assess physical activity habits throughout the study protocol and to ensure subjects complied with their instructions to maintain their physical activity habits. The SF-36 Health Survey was administered before and after 6 weeks of supplementation to assess quality-of-life. The SF-36 has been used in previous intervention and population-based investigations as an assessment of quality-of-life status and provides a physical and mental function score (see McHorney, et al., (1994) MED CARE, 32(1):40-66; McHorney, et al., (1993) MED CARE, 31(3):247-63; Orrell et al., (2017) PLOS MED., 14(3): e1002269; Wayne et al., (2015) J MED INTERNET RES., 17(10): e224).

The GAD-7 was administered before and after 6 weeks of supplementation to assess general anxiety (see Spitzer et al., (2006) ARCH INTERN MED., 166(10):1092-7). The GAD-7 assessed the frequency of 7 items over the previous two weeks (“Feeling nervous, anxious, or on edge,” “Not being able to stop or control worrying,” “Worrying too much about different things,” “Trouble relaxing,” “Being so restless that it's hard to sit still,” “Becoming easily annoyed or irritable,” and “Feeling afraid as if something awful might happen”). A higher score on the GAD-7 indicates a greater degree of anxiety.

Stool Kits: At-home stool sample kits were provided to all subjects at their baseline visit (visit 2) with detailed instructions. Subjects were asked to avoid caffeine 24 hours prior to collection. Subjects were asked to collect and send their baseline stool kit prior to starting the 6-week supplementation period and to collect a sample at the end of the supplementation period. The kits included a swab, collection and transport tube, plastic sample bag, and return mailer envelope. Subjects were instructed to apply the tip of the swab against used toilet paper until the swab was covered in fecal material and then place it in the collection and transport tube for mailing. Kits were coded for blinded analyses upon receipt.

Microbiome analysis; DNA Extraction: DNA was extracted using QIA amp Fast DNA Stool Mini kit (Qiagen GmpH, Hilden, Germany) according to manufacturer's instructions. Fecal samples swabs were transferred to tubes containing 1 ml of InhibitEX lysis Buffer. Swabs were incubated for 1 hour at 75 °C and shaken using Fastprep-96 4 times for 5 min each at a speed of 1200 RPM. An equal amount of 100% ethanol was added to the lysate that was passed through DNA binding columns. DNA was eluted from the columns using 50 μl of molecular grade water. The quality and purity of the isolated genomic DNA was confirmed by gel electrophoresis and quantified using a Qubit 2.0 instrument and the Qubit dsDNA HS Assay (Life Technologies). Purified DNA samples were stored at −20 °C until use.

PCR Amplification: Amplification of the 16S and 5.8S rRNA genes was performed using primers specific for bacterial 16S-515 (5′-(GGA CTA CCA GGG TAT CTA ATC CTG)-3′ (“SEQ ID NO: 1”) and 16S-804 (5′-(TCC TAC GGG AGG CAG CAG T)-3′) (“SEQ ID NO: 2”), and fungal ITS1 (5′-(TCC GTA GGT GAA CCT GCG G)-3′) (“SEQ ID NO: 3”) and ITS4 (5′-TCC TCC GCT TAT TGA TAT GC)-3′) (“SEQ ID NO: 4”), respectively. The PCR mixture contained Q5 High-Fidelity Master Mix (New England Bioinformatics) at a 1× concentration, and 0.05 μl/mM of each primer. Template DNA (100 ng) was added to each 50 μl reaction. Thermo-cycling conditions included an initial denaturation step (3 minutes at 98 °C), followed by 30 cycles of denaturation (10 seconds at 98 °C), annealing (10 seconds at 55 °C for the 16S primers and 20 seconds at 58 °C for the ITS primers), extension (10 seconds at 72 °C), and a final extension step of 3 minutes at 72 °C. PCR products were confirmed using gel electrophoresis on a 1.5% agarose gel (containing 7 μg/ml ethidium bromide).

Library Preparation and Sequencing: The amplicon library was cleaned and barcoded followed by emulsion PCR using an Ion Torrent S5 Prime workflow according to the manufacturer's instructions (ThermoFisher). Equal volumes of bacterial (16S) and fungal (ITS) rRNA amplicons were pooled, cleaned with AMPure XP beads (Beckman Coulter, CA, USA) to remove unused primers, and then exposed to an end repair reaction (ThermoFisher) for 20 minutes at room temperature. After an additional AMPure clean up step, ligation was performed at 25 °C for 30 minutes using an Ion Torrent P1 and a unique barcoded ‘A’ adaptor per pooled sample, following AMPure removal of residual adaptors. All separate barcoded samples were then pooled in equal amounts (10 μl) and size selected for the anticipated 16S and ITS range (200-800 bp) using Pippin Prep (Sage Bioscience). The library was amplified for seven cycles and quantified using a StepOne qPCR instrument prior to dilution to 300 μM and loading into an IonSphere templating reaction for use in an Ion Chef System. Library sequencing was performed using an Ion Torrent S5 sequencer (ThermoFisher).

Bioinformatics: Pre-Processing: De-multiplexing of the sequencing output was performed using Python 2.7 with the input equal to adapter-trimmed output from the Ion Torrent sequencing platform. A map file that links the sample ID to the barcodes, adapted to the amplicons in the ligation phase of the library preparation, was created.

The de-multiplexed data was then parsed for quality (Q20) and sequence lengths sequestered to 200-400/400-800 bp, for 16s/ITS respectively. Finally, the sequences were de-noised and chimeras removed. Operational taxonomic units (OTUs) were generated using de novo clustering by a defined similarity threshold of 0.97. Sequences that were similar, at, or above the accepted threshold level represent the presence of a taxonomic unit (e.g., a species similarity threshold was set at 0.97) in the sequence collection. Taxonomy was assigned using a custom pipeline based on the Greengenes V13_8 and UNITE database V7.2 taxonomic classification of 16s and ITS sequences, respectively. Blastn was used for alignment with an error threshold of 0.001 for selection. Data was summarized, and prepared reads were represented by absolute count per ID. Statistical analysis was performed in R using Packages.

Data Processing: Raw 16S and ITS absolute count matrices were obtained from the pre-processing step, and sample annotation were loaded to R version 4.0.3. Using R package microbiome 1.12.0 and phyloseq 1.34.0, a phyloseq object containing a read count matrix (species level identification OTUs), taxonomic table (kingdom down to species) and sample annotation was constructed. Initially the 16S and ITS were handled separately until after the read counts were normalized and transformed to relative abundance.

Data Cleaning and QC: OTU identifiers were used to construct the taxonomic table. The table was cleaned by removing OTUs that were annotated as “unknown/unidentified” at a phyla, genus or species level. This step ensured only species that were identifiable were analyzed. The quality of the samples was assessed using the total read count after the taxonomic table had been cleaned and aggregated to a species level. A minimum of 500 16S read counts was used as the lower cut-off. Finally, samples with a read count <500 were removed, and the data was normalized and transformed to relative abundance.

Data exploration: Composition bar graphs were generated using 16S and ITS relative abundance data separately, after aggregating the data to the phyla level. Filtering on detection limit (0.001% minimum) and prevalence (10% amongst all samples) was used. These graphs present a simple overview of the phyla abundances in the samples.

Ordinate analysis/compositional principal component analysis (PCA) was performed on 16S and ITS data separately, using species level relative abundances. Filtering on detection limit (0.00001% minimum) and prevalence (20% amongst all samples) was performed. Ordinate analysis was done using the “ordinate( )” function with method “MDS” (multidimensional scaling) and “Bray-Curtis” distance, from R package microbiome version 1.12.0. Alpha diversity measures were calculated using the “alpha( )” function with “Shannon index” as the diversity measure, from R package microbiome version 1.12.0.

Data Analysis: Because the same subject had a Pre- and Post-time point data, the Wilcoxon-rank-sum test “wilcox.test( )” with option “paired=TRUE” from R package stats version 4.0.3 for comparisons from the same treatment group (PRO/PLA) was used. For comparisons between PRO and PLA groups, a “wilcox.test( )” with option “paired=FALSE” was used. A significance level of P≤0.05 was accepted as statistically significant, while a significance level of P>0.051 to ≤0.1 was accepted as a statistical trend. Fold-change between groups was calculated using mean relative abundance of the taxa using the “foldchange( )” function from R package gtools version 3.9.2. All figures related to this data were generated using ggplot2 and ggpubr R packages versions 3.3.5 & 0.4.0, respectively.

Statistical Analyses for non-microbiome related variables: Normality of each variable was assessed using the Shapiro-Wilk test. Although a few variables at various time points did exhibit a non-normal distribution, traditional ANOVAs are relatively robust in response to non-normality. Thus, 2 (PRO vs. PLA)×2 (week 0-PRE, week 6-POST) mixed factorial ANOVAs were completed to assess group, time, and group×time interaction effects for all variables. When sphericity was violated, Greenhouse-Geisser corrected P-values were used for main effects/interactions. In the event of missing data, a mixed-effects model was utilized in GraphPad Prism. Sidak post-hoc procedures were used to assess individual comparisons between time points and/or groups. A significance level of ≤0.05 was accepted as statistical significance, whereas a significance level of >0.051 to ≤0.10 was accepted as a statistical trend. Trends and significant main effects/interactions were followed up with dependent t-tests to identify specific differences. For between-group changes over time, independent t tests (for data displaying normal distribution assessed via Shapiro-Wilk test) and Mann-Whitney tests (for data not displaying normal distribution assessed via Shapiro-Wilk test) were used to assess the change score (deltas) on all variables. Delta values were computed by the differences in time points relative to baseline (i.e., Post-Pre). All data points less than −3 standard deviation (SD) or greater than +3 SD were deemed outliers and thus removed before analyses. Effect of sizes are expressed as Cohen's d with 95% confidence intervals and interpreted as ≥0.2 for a small effect, ≥0.5 for a moderate effect, and ≥0.8 for a large effect. All analyses were completed with GraphPad Prism version 9.2.0 (GraphPad Software, San Diego, CA, USA).

Results-Microbiome analyses: Regarding the gut microbiota composition, stool samples at baseline (Pre) and after the intervention (Post) were collected from 24 subjects assigned to the probiotic treatment group (PRO) or 27 subjects assigned to the placebo control treated cohort (PLA). Subjects provided samples for 16s (for the bacterial community) and ITS (for the fungal community) rRNA gene sequencing. Following internal read count quality control (QC), 18 (Pre) and 21 (Post) samples from the PRO cohort and 23 (Pre) and 21 (Post) samples from the PLA cohort were analyzed for total gut microbiota comparisons. Furthermore, in order to have a better understanding of the intervention impact on the function of microbiome, a subset of the PRO and PLA cohort individuals (n=17 and 19, respectively) who provided matched Pre- and Post-samples that passed internal QC on total read counts was analyzed.

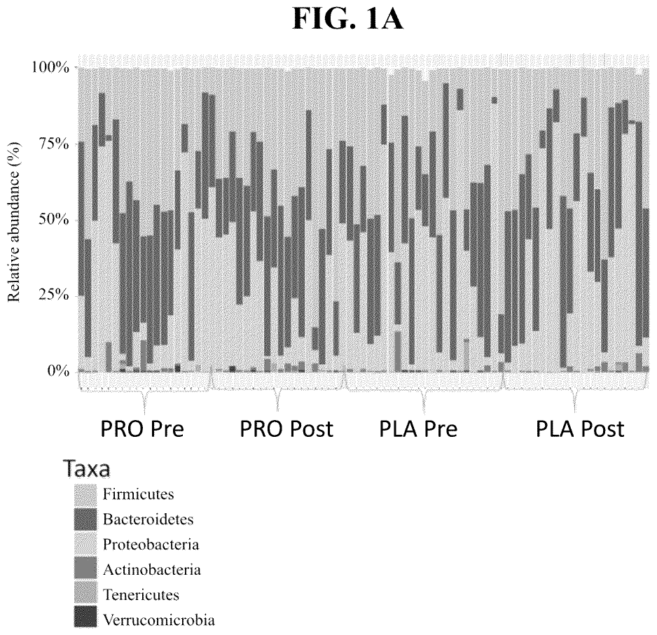

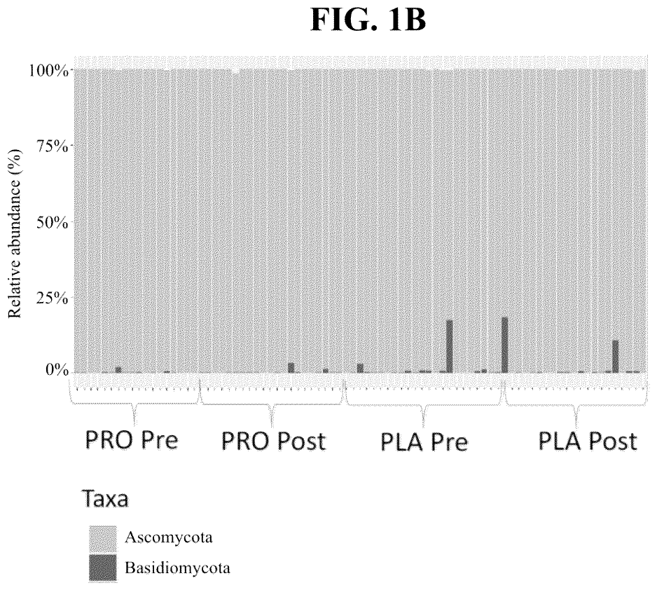

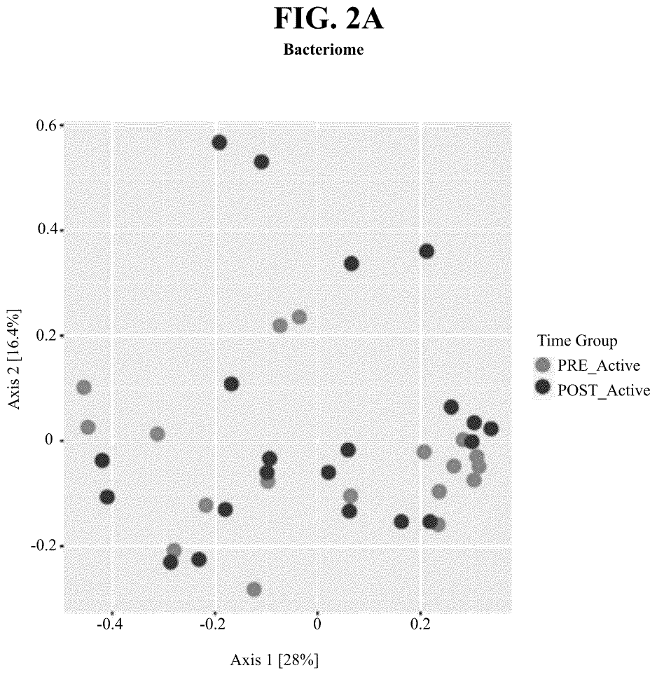

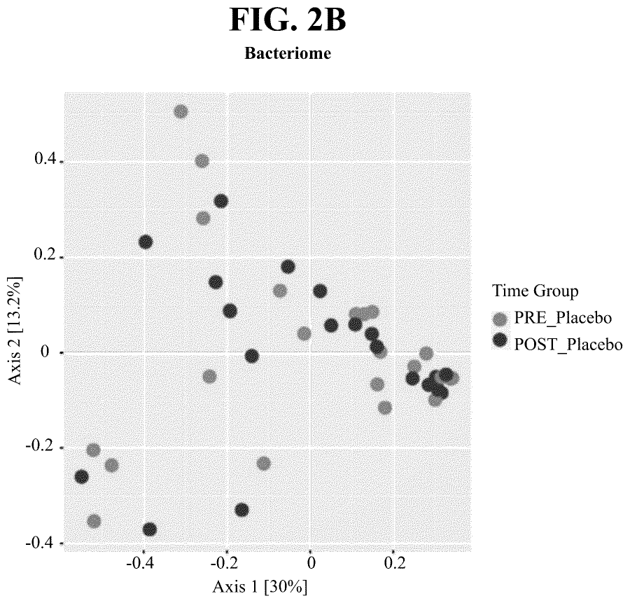

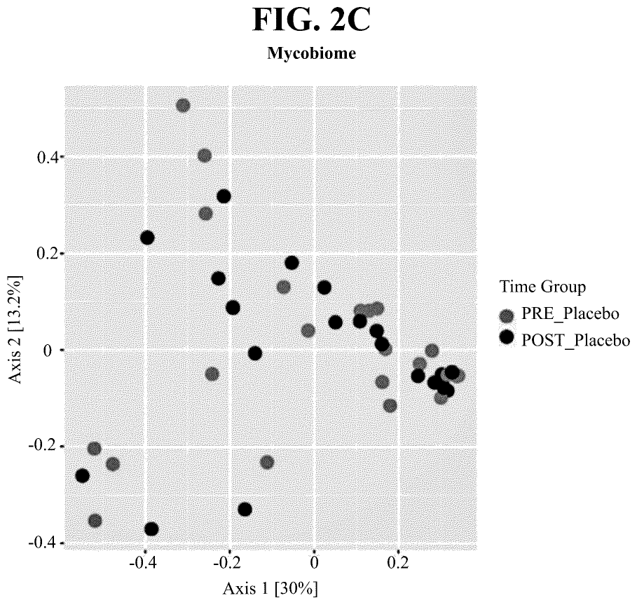

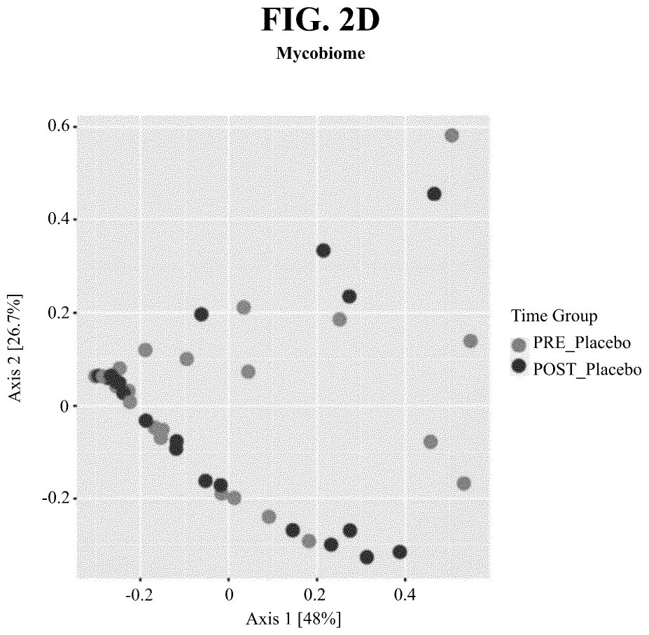

Compositional differences in gut microbiota between treatment groups at the Phyla level are presented in FIGS. 1A-1B. The average relative abundances of bacterial and fungal phyla detected in stool samples from subjects consuming either PRO or PLA before (Pre) and after (Post) treatment. Multi-Dimensional Scaling (MDS) of Bray-Curtis similarity between OTU profiles from distinct component analysis of gut bacteria (FIGS. 2A-2B) and fungi (FIGS. 2C-2D) composition of subjects before and after (Pre, Post) treatment (PRO, PLA) are presented. The data showed that there is no clear clustering of samples based on any one individual treatment group. As can been seen in FIGS. 2A-2D, the PRE-PRO and POST-PRO samples tend to separate more for both bacterial and fungal groups, indicating potential differences between the samples following PRO treatment.

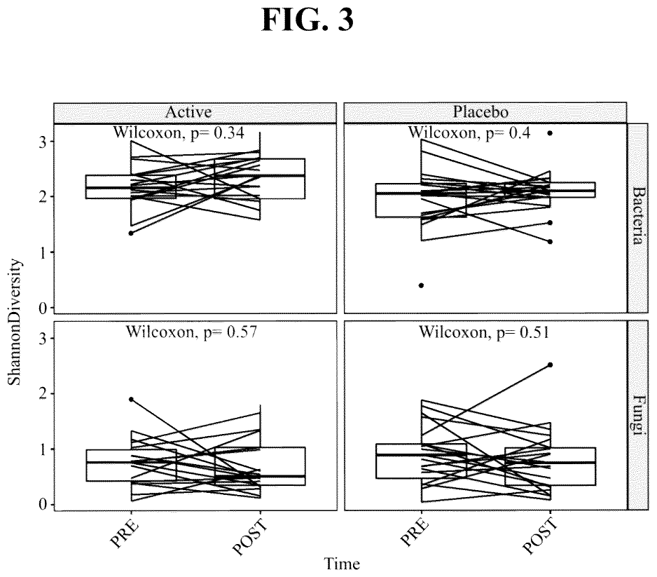

Alpha Diversity of the bacterial and fungal biomes was calculated for each sample that passed QC. As seen in FIG. 3, there were no statistically significant differences between Pre and Post time points in either the PRO or PLA cohort.

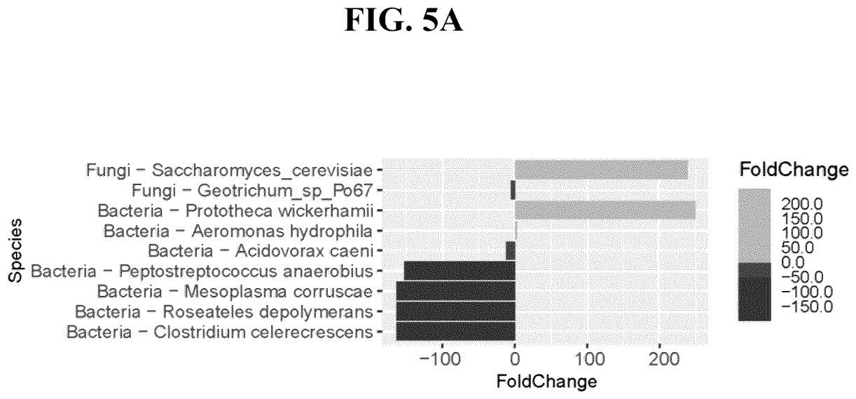

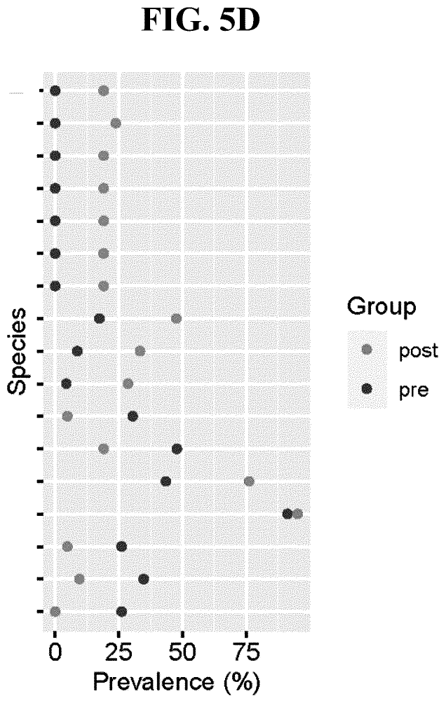

Using unpaired abundance analysis, a number of organisms (bacteria and fungi) that were significantly altered (Pre versus Post) in the PRO and PLA treatment cohorts were found (FIGS. 5A-5B). The taxa shown in FIG. 5A-5B are significantly different between the Pre-versus Post samples of the PRO cohort (P<0.05). There is a significant rise in the abundance (200-fold) of Saccharomyces cerevisiae (FIG. 5A), as well as an increase in its prevalence (FIG. 5B) amongst members of the PRO cohort (~20% to ~60%). For the PLA treated cohort (FIG. 5C-5D), most changes in abundance were less than 50-fold in Pre and Post timepoints, with some organisms increasing, while others decrease. Among fungal organisms, there was a modest increase in S. cerevisiae in the PLA group compared to the PRO treated cohort, and a 50-fold decrease in Candida parapsilosis (a known skin colonizer (see Kuhbacher et al. (2017) MICROORGANISMS. 2017; 5(2)). Interestingly, among the most highly downregulated species in the PLA treatment cohort, Bacillus thuringiensis was decreased more than 150-fold in abundance with a concomitant decrease in prevalence. This organism is a highly utilized environmentally compatible biologic pesticide that may be detected due to consumption of food (see Jouzani et al. (2017) APPL MICROBIOL BIOTECHNOL. 2017; 101(7):2691-711). The organism with the most dramatic decrease among the PLA cohort, Macrococcus caseolyticus, was decreased 175-fold with a decrease in prevalence also noted. This organism is normally reported to colonize the skin of various mammals and may be introduced through food products derived from them, such as raw and pasteurized milk, dairy products, and meats (see Cicconi-Hogan et al. (2014) J DAIRY SCI. 2014; 97(5):2959-64; Mazhar et al. (2020) FRONTIERS IN MICROBIOLOGY. 2020; 11:1533).

When comparing PRO versus PLA in the Post-treatment group only, significantly different abundance of bacterial and fungal species are found between the two groups (P<0.05). However, many of the fungi identified were listed as uncultured and may be due to poor taxonomic identification potential or potentially transient detection based on the observation of Hallen-Adams (see Hallen-Adams et al. (2017) VIRULENCE 8(3):352-8). Many of the other increased fungal organisms (e.g., Waitea circinata, Aspergillus foetidus, Issatchenkia_terricola) have been associated with food products or treatment. There was an observed significant decrease in the pathogenic organism Listeria monocytogenes in the PRO versus PLA cohort, potentially a good indicator of improved gut health (FIGS. 4A-4B).

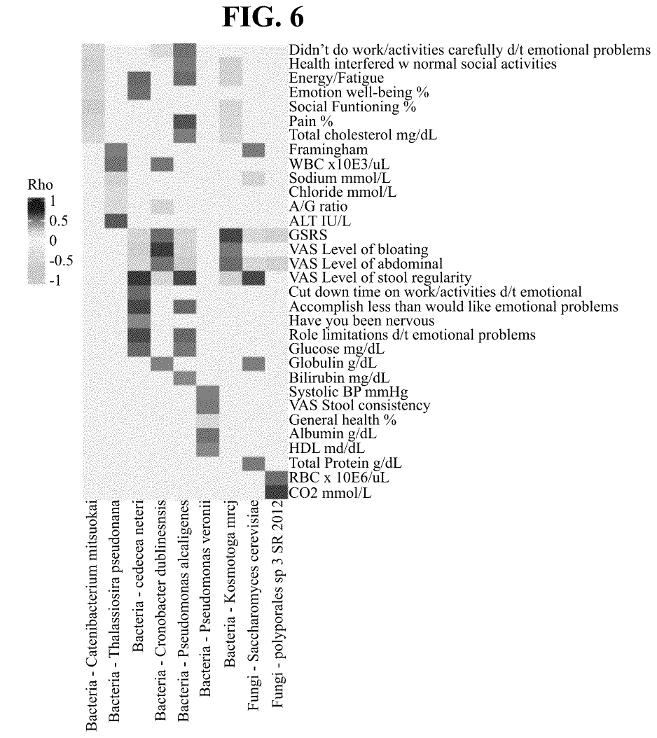

In order to determine if there were further associations between clinical features and the gut microbiota abundance, a Spearman rank correlation (Rho) analysis to assess the strength of association between microbiome and clinical biomarkers was performed (FIG. 6). Based on the analysis, 9 species (7 bacterial and 2 fungal) that were significantly (negatively or positively) associated with the change in 32 clinical features were identified.

Quality of Life Measurements (Framingham, GAD-7, SF-36): No differences were noted in Framingham physical activity score pre and post intervention (PRO: 35.6±5.2 to 35.7±5.2 au vs. PLA: 35.6±6.5 to 36.6±7.0 au, respectively). 30 subjects completed the generalized anxiety disorder (GAD-7) questionnaire. The assessment before (Pre) and after (Post) active probiotic (PRO) and placebo (PLA) consumption is provided in TABLE 7.

TABLE 7

| Variable | Time | PRO | PLA |
|---|---|---|---|
| Nervous/anxious | Pre | 1.4 ± 1.0\hat{ } | 0.6 ± 0.6 |
| (au) | Post | 0.7 ± 0.6 | 0.3 ± 0.5^{#} |
| Control worrying | Pre | 0.8 ± 1.0^{β} | 0.1 ± 0.4 |
| (au) | Post | 0.4 ± 0.5 | 0.3 ± 0.6 |
| Worrying too much | Pre | 1.0 ± 0.9 | 0.4 ± 0.5 |
| (au) | Post | 0.7 ± 0.7 | 0.6 ± 0.7 |
| Trouble relaxing (au) | Pre | 1.1 ± 1.1 | 0.8 ± 0.9 |
| | Post | 0.7 ± 0.6 | 0.6 ± 0.7 |
| Restless (au) | Pre | 0.8 ± 1.0 | 0.7 ± 1.2 |
| | Post | 0.5 ± 0.6 | 0.5 ± 0.6 |
| Irritable (au) | Pre | 1.4 ± 1.1 | 1.1 ± 1.0 |
| | Post | 0.7 ± 0.8* | 1.0 ± 0.9 |
| Feeling of Doom (au) | Pre | 0.5 ± 0.8\hat{ } | 0.1 ± 0.3 |
| | Post | 0.3 ± 0.6 | 0.1 ± 0.4 |
| Total score (au) | Pre | 7.1 ± 5.8 | 3.9 ± 3.8 |
| | Post | 4.0 ± 3.4^{#} | 3.5 ± 3.7 |

Table 7 shows General Anxiety Disorder (GAD-7) assessments before (Pre) and after (Post) active probiotic (PRO) and placebo (PLA) consumption. Data are shown as Mean ± SD. PRO = Probiotic group; PLA = Placebo group.
*Significantly different from Pre (p ≤ 0.05).
^{#}Trend from Pre (p ≤ 0.10).
^{β}Trend from pre PLA (p ≤ 0.10).
\hat{ }Significantly different from pre PLA (p ≤ 0.05).

There was a significant main effect of time (Pre-versus Post-) (P=0.024) and a trend for an interaction (P=0.073) on the GAD-7 total score. Post-hoc testing indicated a trend for differences between Pre- and Post-within the PRO treated cohort (P=0.054) (Post was lower than Pre indicating a possible improvement in GAD-7 scores by ~44% relative to baseline in the PRO treated cohort). In addition, the change score/delta between groups for total score on the GAD-7 indicated significant differences (P=0.041). On average PRO had a larger reduction relative to baseline in their GAD-7 total score than PLA (−3.1±4.8 (~44%) vs. −0.4±2.3 au (~10%), respectively).

There was a significant main effect of time (p=0.016) and group (p=0.006) along with a trend for an interaction (p=0.079) on “Feeling nervous, anxious, or on edge.” Post hoc testing indicated a significant difference between pre and post for PRO (p=0.038) and PLA (p=0.034) showing that pre was greater than post by ~48% in PRO and ~52% in PLA relative to baseline (showing improvement in both groups). In addition, pre PRO was significantly greater than pre PLA (p=0.011) (indicating differences at baseline) and there was a trend for differences between groups at post (p=0.083) suggesting PRO may have been greater than PLA. There was a significant main effect for group (p=0.034) and a trend for an interaction (p=0.068) for “Not being able to stop or control worrying.” Post hoc testing indicated a trend between PRO and PLA at pre (p=0.054) with PRO being greater than PLA (showing possible differences between groups at baseline). In addition, the change score/delta between groups for “Not being able to stop or control worrying” indicated a trend for differences (p=0.058). On average PRO may have had a reduction in their feeling of “Not being able to stop or control worrying” compared to a possible slight increase in PLA (−0.4±0.8 vs. 0.1±0.6 au, respectively). There was a trend for an interaction (p=0.085) for “Worrying too much about different things,” however there were no post hoc differences. There was a significant main effect of time (p=0.049) for “Trouble relaxing,” however there were no post hoc differences. There was a trend for time (p=0.070) for “Being so restless that it's hard to sit still,” however there were no post hoc differences. There was a trend for time (p=0.077) and a significant interaction (p=0.048) for “Becoming easily annoyed or irritable.” Post hoc testing indicated that post was significant less than pre for PRO (p=0.025). In addition, the change score/delta between groups for “Becoming easily annoyed or irritable” indicated a trend for differences (p=0.088). On average PRO may have had a larger reduction in becoming easily annoyed or irritable than PLA (−0.7±0.9 vs. −0.1±1.0 au, respectively). There was a group trend (p=0.084) for “Feeling afraid as if something awful might happen.” Post hoc testing indicated a difference at pre between groups (p=0.034).

For the health survey questionnaire (SF-36) multiple differences in PRO versus PLA were observed and are summarized in TABLE 8.

TABLE 8

| Variable | Time | PRO | PLA |
|---|---|---|---|
| Cut down time on | Pre | 83.3 ± 38.1 | 96.2 ± 19.6 |
| work/activities d/t | Post | 96.0 ± 20.0 | 92.6 ± 26.7 |
| emotional problems | | | |
| (au) | | | |
| Accomplish less than | Pre | 70.8 ± 46.4 | 84.6 ± 36.8 |
| would like d/t | | | |
| emotional problems | Post | 84.0 ± 37.4 | 85.2 ± 36.2 |
| (au) | | | |
| Didn't do | Pre | 83.3 ± 38.1 | 100.0 ± 0.0 |
| work/activities | | | |
| carefully d/t | | | |
| emotional problems | Post | 100.0 ± 0.0 | 96.3 ± 19.2 |
| (au) | | | |
| Health interfered w | Pre | 79.2 ± 28.2 | 93.3 ± 13.3 |
| normal social | | | |
| activities (au) | Post | 93.0 ± 11.5# | 90.7 ± 15.7 |
| Have you been | Pre | 75.0 ± 30.8 | 80.0 ± 18.8 |
| nervous (au) | Post | 76.0 ± 22.4 | 81.5 ± 16.6 |
| Calm and peaceful | Pre | 63.3 ± 27.5 | 66.9 ± 26.5 |
| (au) | Post | 64.8 ± 21.8 | 68.1 ± 23.7 |
| Physical functioning | Pre | 89.0 ± 19.5 | 87.1 ± 19.6 |
| (%) | Post | 92.8 ± 10.8 | 93.3 ± 9.6 |
| Role limitations d/t | Pre | 77.1 ± 38.2 | 87.5 ± 26.7 |
| physical health (%) | Post | 89.0 ± 24.0 | 84.3 ± 26.1 |
| Role limitations d/t | Pre | 79.2 ± 37.8 | 93.6 ± 13.4 |
| emotional health (%) | Post | 93.3 ± 16.7 | 91.4 ± 23.7 |
| Energy/Fatigue (%) | Pre | 55.6 ± 23.1 | 63.3 ± 20.7 |
| | Post | 66.0 ± 13.7* | 62.8 ± 19.6 |
| Emotional well-being | Pre | 76.5 ± 20.0 | 80.0 ± 15.1 |
| (%) | Post | 81.1 ± 14.1 | 83.1 ± 12.6 |
| Social functioning | Pre | 81.8 ± 25.0 | 91.8 ± 15.8 |
| (%) | Post | 91.5 ± 13.8# | 90.7 ± 14.9 |
| Pain (%) | Pre | 74.7 ± 26.6 | 84.0 ± 21.6 |
| | Post | 85.5 ± 17.6# | 82.3 ± 18.3 |
| General health (%) | Pre | 74.8 ± 14.3 | 78.1 ± 18.0 |
| | Post | 79.8 ± 16.6* | 81.3 ± 19.1 |
| Health Change (%) | Pre | 50.0 ± 25.5 | 58.7 ± 21.1 |
| | Post | 61.0 ± 24.0 | 56.5 ± 22.6 |

Table 8 shows 36-Item Short Form Health Survey (SF-36) assessments before (Pre) and after (Post) active probiotic (PRO) and placebo (PLA) consumption to address health-related quality of life responses. Data are shown as Mean ± SD. PRO = Probiotic group; PLA = Placebo group.
*Significantly different from Pre (p ≤ 0.05).
#Trend from Pre (p ≤ 0.10).

For overall heath indicators, there was a trend (P=0.053) for the category “General Health.” Post-hoc testing indicated that post was significantly greater than pre in PRO (P=0.039) showing an improvement in General Health after the PRO intervention. There was a trend for an interaction (P=0.066) in the category “Health Change,” however there were no post-hoc differences. However, the delta (change in score) between groups for “Health change” indicated a trend for differences (P=0.075). On average PRO may have had an improvement while PLA may have had a worse score from pre/baseline (11.5±26.6 vs. −1.9±25.4 au, respectively).

There was a significant interaction (p=0.016) for the question “Didn't do work/activities carefully due to emotional problems,” however there were no post-hoc differences. In addition, the delta (change in score) between groups for “Didn't do work/activities carefully due to emotional problems” indicated significant differences (p=0.019). On average PRO had an improvement (by ~20%) while PLA had a poorer score (by ~4%) from pre/baseline (16.7±38.1 vs. −3.9±19.6 au, respectively). There was a significant interaction (p=0.013) and a trend for time (p=0.063) for “Health interfered with normal activities.” Post hoc testing indicated a trend from pre to post within PRO (p=0.059) showing a possible improvement after the intervention. In addition, the change score/delta between groups for “Health interfered with normal social activities” indicated a significant difference (p=0.012). On average PRO had an improvement while PLA had a worse score from pre/baseline (13.5±24.4 vs. −1.9±17.2 au, respectively). There was a significant main effect of time (p=0.032) for the composite score of physical functioning, however there were no post hoc differences. There was a trend for an interaction for “Role limitations due to emotional health.” however there were no post hoc differences. There were trends for time (p=0.100) and an interaction (p=0.075) for Energy/Fatigue. Post hoc testing indicated that post was significantly greater than pre in PRO (p=0.012) showing a possible improvement after the intervention. There was a trend for time (p=0.070) for “emotional well-being,” however there were no post hoc differences. There was a significant interaction (p=0.048) and a trend for time (p=0.085) for Social Functioning. Post hoc testing indicated a trend for PRO indicating that post may have been greater than pre (p=0.052) showing a possible improvement. In addition, the change score/delta between groups for Social functioning indicated a trend for differences (p=0.052). On average PRO may have had an improvement while PLA may have had a worse score from pre/baseline (9.4±20.3 vs. −0.5±14.4 au, respectively). There was a significant interaction (p=0.047) for Pain. Post hoc testing indicated a trend between pre and post for PRO (p=0.055) showing a possible larger value at post and thus a possible improvement in Pain after the intervention. There were no differences noted in “Cut down time on work/activities due to emotional problems,” “Accomplish less than would like due to emotional problems,” “Have you been nervous,” “Calm and peaceful,” “Role limitations due to physical health.”

Safety screen bloodwork (CBC, CMP, Lipid panel) and adverse events (AEs): Although some parameters of the CBC values showed statistically significant differences from pre-post, or between groups, all values remained within the normal clinical laboratory reference range. There were no differences noted in WBC, BUN, BUN/creatinine ratio, Sodium, Potassium, Chloride, CO2, total protein, albumin, A/G ratio, Bilirubin, ALK phosphate, AST, total cholesterol, triglycerides, VLDL, or LDL. (TABLE 9). Subjects in both groups tolerated PRO and PLA well with ~10% of subjects in both groups reporting possible grade 1 adverse events to either test article including: mild abdominal pain, vivid dreams, a maculo-papular rash, vertigo, and headache.

TABLE 9

| Variable | Time | PRO | PLA |
|---|---|---|---|
| WBC (x10 E3/uL) | Pre | 6.2 ± 1.8 | 5.5 ± 2.0 |
| | Post | 5.8 ± 1.6 | 5.5 ± 1.9 |
| RBC (x10 E6/uL) | Pre | 4.6 ± 0.4 | 4.5 ± 0.5 |
| | Post | 4.8 ± 0.4* | 4.6 ± 0.5* |
| Hgb (g/dL) | Pre | 14.1 ± 1.4 | 14.2 ± 1.6 |
| | Post | 14.6 ± 1.5* | 14.4 ± 1.6^{#} |
| Hct (%) | Pre | 41.6 ± 3.5 | 41.4 ± 4.1 |
| | Post | 42.5 ± 4.1 | 41.5 ± 3.7 |
| Glucose (mg/dL) | Pre | 88.9 ± 9.3 | 88.4 ± 6.6 |
| | Post | 91.6 ± 11.7 | 90.4 ± 7.2 |
| BUN (mg/dL) | Pre | 13.1 ± 3.6 | 13.0 ± 3.4 |
| | Post | 13.1 ± 4.2 | 12.7 ± 3.4 |
| Creatinine (mg/dL) | Pre | 0.86 ± 0.13 | 0.79 ± 0.14 |
| | Post | 0.88 ± 0.20 | 0.83 ± 0.15 |
| BUN/Creatinine ratio | Pre | 15.5 ± 5.1 | 16.8 ± 4.8 |
| | Post | 15.4 ± 5.8 | 15.0 ± 4.0 |
| Sodium (mmol/L) | Pre | 140.2 ± 1.9 | 139.4 ± 2.1 |
| | Post | 140.0 ± 1.8 | 139.6 ± 1.7 |
| Potassium (mmol/L) | Pre | 4.3 ± 0.2 | 4.3 ± 0.3 |
| | Post | 4.3 ± 0.3 | 4.3 ± 0.2 |
| Chloride (mmol/L) | Pre | 102.7 ± 2.0 | 102.8 ± 2.3 |
| | Post | 102.5 ± 2.2 | 102.7 ± 2.1 |
| CO2 (mmol/L) | Pre | 22.5 ± 1.8 | 23.1 ± 1.6 |
| | Post | 22.8 ± 1.9 | 22.8 ± 1.0 |
| Calcium (mg/dL) | Pre | 9.4 ± 0.4 | 9.4 ± 0.3 |
| | Post | 9.4 ± 0.3 | 9.4 ± 0.3 |
| Total protein (g/dL) | Pre | 6.9 ± 0.4 | 7.0 ± 0.3 |
| | Post | 7.0 ± 0.4 | 7.1 ± 0.4 |
| Albumin (g/dL) | Pre | 4.5 ± 0.2 | 4.6 ± 0.3 |
| | Post | 4.6 ± 0.3 | 4.7 ± 0.3 |
| Globulin (g/dL) | Pre | 2.4 ± 0.3 | 2.4 ± 0.3 |
| | Post | 2.4 ± 0.3 | 2.5 ± 0.4 |
| A/G ratio | Pre | 1.9 ± 0.3 | 2.0 ± 0.3 |
| | Post | 1.9 ± 0.3 | 1.9 ± 0.3 |
| Bilirubin (g/dL) | Pre | 0.5 ± 0.2 | 0.5 ± 0.2 |
| | Post | 0.5 ± 0.3 | 0.5 ± 0.3 |
| ALK Phosphate | Pre | 70.1 ± 17.7 | 69.9 ± 17.6 |
| (IU/L) | Post | 76.7 ± 19.8 | 71.6 ± 19.7 |
| AST (IU/L) | Pre | 23.4 ± 9.6 | 21.5 ± 5.5 |
| | Post | 23.3 ± 12.7 | 23.5 ± 8.4 |
| ALT (IU/L) | Pre | 27.2 ± 14.9 | 21.8 ± 13.2 |
| | Post | 27.0 ± 18.8 | 24.2 ± 15.7 |
| Total cholesterol | Pre | 193.5 ± 36.9 | 196.9 ± 37.5 |
| (mg/dL) | Post | 198.2 ± 43.0 | 196.8 ± 25.9 |
| Triglycerides (mg/dL) | Pre | 99.9 ± 38.2 | 96.8 ± 43.8 |
| | Post | 101.8 ± 35.2 | 87.6 ± 30.5 |
| HDL (mg/dL) | Pre | 54.8 ± 12.8 ^{β} | 64.5 ± 17.3 |
| | Post | 58.3 ± 16.2 ^{β} | 68.2 ± 22.6^{#} |
| VLDL (mg/dL) | Pre | 18.0 ± 6.5 | 18.8 ± 9.5 |
| | Post | 18.2 ± 6.1 | 15.7 ± 5.2 |
| LDL (mg/dL) | Pre | 119.4 ± 33.8 | 113.6 ± 34.7 |
| | Post | 121.7 ± 35.1 | 111.7 ± 26.6 |
| LDL/HDL ratio | Pre | 2.3 ± 0.8 | 1.9 ± 0.8 |
| | Post | 2.2 ± 0.8 | 1.8 ± 0.8 |

Table 9 shows safety screen bloodwork (Routine Complete Blood Chemistry (CBC) before (Pre) and after (Post) active probiotic (PRO) and placebo (PLA) consumption. Data are shown as Mean ± SD. PRO = Probiotic group; PLA = Placebo group.
*Significantly different from Pre (p ≤ 0.05).
^{#}Trend from Pre (p ≤ 0.10).
^{β} Trend from PLA (p ≤ 0.10).

Blood pressure (BP), heart rate (HR), and body weight (BW): Despite some statistically significant differences from pre-post, or between groups, vital signs remained within the normal clinical laboratory reference range for the duration of the study. For individual parameters, a significant effect for time on diastolic blood pressure (DBP) was noted (P=0.030). Post-hoc comparisons indicated that Post was significantly greater than Pre for PRO treatment (~3 mmHg, P=0.027). There was a trend in time for systolic blood pressure (SBP) as well (P=0.078). There was an observed significant interaction for HR (P=0.044), however there were no post-hoc differences. Moreover, a significant main effect of time (P=0.001) for body weight was also noted. Post-hoc testing revealed that visit 1 screening body weight for PRO was potentially lower at post (mean difference=0.8 kg, P=0.095, d=−0.45) indicating a possible gain in weight. Similarly, body weight at visit 1 screening (mean difference=0.7 kg, P=0.025, d=−0.55) and visit 2 baseline/pre (mean difference=0.7 kg, P=0.031, d=−0.53) were both significantly lower than visit 3 post for PLA indicating weight gain. These differences are summarized in TABLE 10.

There was a significant main effect of time for RBC (p=0.003). Post-hoc testing indicated post was significantly greater than pre in PRO (p=0.019, d=−0.57) and PLA (p=0.020, d=−0.37) while staying within the normal clinical reference range. There was a significant main effect of time for Hgb (p=0.009). Post-hoc testing indicated Post was significantly greater than Pre in PRO (p=0.008) while a trend was noted in PLA (p=0.069) showing that post may be greater than pre. There was a significant main effect of time for Hct (p=0.032), however there were no post-hoc differences. There was a significant main effect of time for fasting blood glucose (p=0.030), however there were no post-hoc differences. There was a trend for group (p=0.098) in creatinine, however there were no post-hoc differences. In addition, the change score/delta between groups for creatinine indicated a significant difference (p=0.022). On average, PRO stayed the same while PLA had a slightly higher level than pre/baseline (0.00±0.70 vs. 0.03±0.06 mg/dL, respectively). There was a trend for an interaction (p=0.099) in calcium, however there were no post-hoc differences. In addition, the change score/delta between groups for calcium indicated a trend for differences (p=0.085). On average PRO may have had a reduction while PLA may have stayed the same compared to pre/baseline (−0.1±0.3 vs. 0.0±0.3 mg/dL, respectively). There was a trend for time for globulin (p=0.065), however there were no post-hoc differences. There was a trend for group for ALT (p=0.093), however there were no post-hoc differences. In addition, the change score/delta between groups for ALT indicated a significant difference (p=0.018). On average PRO had a decrease while PLA had an increase in levels compared to pre/baseline (−1.3±9.1 vs. 2.4±6.7 IU/L, respectively). There was a significant main effect of time (p=0.010) and group (p=0.045) for HDL. Post-hoc analysis indicated a trend from pre to post in PLA (p=0.060) showing that post may have been greater than pre. In addition, there were trends between groups at pre (p=0.068) and at post (p=0.058) indicating that PLA may have been greater than PRO before and after the intervention. There was a trend for group (p=0.093) for LDL/HDL ratio, however there were no post-hoc differences.

TABLE 10

| Variable | Time | PRO | PLA |
|---|---|---|---|
| SBP (mmHg) | Screen | 125 ± 10 | 122 ± 12 |
| | Pre | 123 ± 13 | 125 ± 12 |
| | Post | 127 ± 11# | 126 ± 12 |
| DBP (mmHg) | Screen | 80 ± 7 | 79 ± 7 |
| | Pre | 78 ± 8 | 80 ± 8 |
| | Post | 81 ± 8* | 81 ± 8 |
| HR (bpm) | Screen | 75 ± 10 | 70 ± 9 |
| | Pre | 75 ± 10 | 71 ± 10 |
| | Post | 72 ± 11 | 73 ± 8 |
| BW (kg) | Screen | 85.5 ± 18.6\hat{ } | 78.7 ± 18.0^{†} |
| | Pre | 85.7 ± 18.5 | 78.7 ± 17.9^{†} |
| | Post | 86.3 ± 18.8 | 79.4 ± 18.3 |

Table 10 shows vitals (systolic and diastolic blood pressure, resting heart rate) and body weight measures before (Pre) and after (Post) active probiotic (PRO) and placebo (PLA) consumption. Data are shown as Mean ± SD. PRO = Probiotic group; PLA = Placebo group.
*Significantly different from Pre (p ≤ 0.05).
#Trend from Pre (p ≤ 0.10).
\hat{ }Trend from post (p ≤ 0.10).
^{†}Significantly different from post (p ≤ 0.05).

Given the increased awareness of the gut microbiome and its impact on digestive health and immune function there is a demand for further clinical investigations regarding the role of probiotics in addressing dysbiosis in different health conditions. This study examined the effects of a probiotic supplement (PRO) on quantified digestive symptoms and modulation of the microbiome structure. These examples showed that the novel probiotic blend (PRO) can improve irritability while also reducing anxiety, reducing emotional problems or worry, and improving overall score on an anxiety symptom questionnaire (GAD-7). Simultaneously, PRO can improve a variety of self-reported conditions related to overall health such as, energy/fatigue, pain, social function, and general health which may have allowed subjects to also report having less interference from health ailments and emotional disturbances in their daily lives (as measured by the SF-36). Lastly, PRO can mitigate weight gain compared with PLA. Importantly, the probiotic supplement (PRO) was well tolerated in individuals in comparison to PLA. Moreover, consumption of PRO led to positive changes in the gut microbiome specifically leading to a highly significant increase in the abundance of S. cerevisiae, a known marker of a healthy gut microbiome (see Abid et al., (2022) J FUNGI (BASEL); 8(5); Garcia et al, (2016) BENEF MICROBES. 7(5):659-68; Sun et al, (2021) FRONT IMMUNOL. 2021; 12:777665).

The gut-microbiota and the brain communicate through a variety of different pathways that offer crosstalk to multiple organs and influences the behavior of the host (see Cryan et al. (2019) PHYSIOL REV. 99(4):1877-2013). Probiotic strains may modulate the microbiome structure including the hypothalamic-pituitary-adrenal (HPA) axis (leading to potential benefits supporting mental health) and the amygdala influencing social behavior (see Id.). In the current investigation, PRO was potentially beneficial in reducing symptoms of stress and anxiety to a greater degree than PLA.

This study demonstrated that the consumption of the novel probiotic (PRO) blend was well-tolerated with very mild side-effects. Ingestion of PRO showed improvement in behavioral (anxiety, worry, irritability) symptoms and overall health (i.e., improved energy, less fatigue, improved pain, and general health) as measured by the validated SF-36 instrument.

The entire disclosure of each of the patent and scientific documents referred to herein is incorporated by reference herein for all purposes.

1. A method of alleviating an anxiety disorder or one or more symptoms thereof in a subject in need thereof, the method comprising:
consuming by or administering to, the subject a composition comprising (i) an isolated and viable non-pathogenic fungal strain, and (ii) an isolated and viable non-pathogenic bacterial strain, thereby to alleviate the anxiety disorder or one or more symptoms thereof in the subject
wherein the non-pathogenic bacterial strain is at least two strains selected from: Lactobacillus rhamnosus, Bifidobacterium breve, Lactobacillus acidophilus, Bifidobacterium bifidum, Lactobacillus reuteri and combinations thereof; and
wherein the non-pathogenic fungal strain is at least one strain selected from: Saccharomyces boulardii, Saccharomyces cerevisiae, Saccharomyces sp HZ178, Saccharomyces bayanus, Pichia burtonii, Pichia jadinii, Pichia kudriavzevii, Pichia onychis, Pichia sp., and Picoa juniper.

2. The method of claim 1 wherein the method is effective to alleviate the one or more symptoms of the anxiety disorder in the subject.

3. The method of claim 1, wherein the composition is freeze dried or spray dried.

4. The method of claim 1, further comprising an enzyme capable of disrupting a biofilm and/or breaking down fiber in the subject.

5. The method of claim 4, wherein the enzyme is selected from the group consisting of amylase, cellulase, hemicellulase, lysozyme, pectinase, DNase I, Serratia peptidase or Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, and phytase.

6. The method of claim 5, wherein the enzyme is an amylase selected from the group consisting of Bacillus stearothermophilus amylase, Bacillus amyloliquefaciens amylase, Bacillus subtilis amylase, Bacillus licheniformi amylase, Aspergillus niger amylase, and Aspergillus oryzae amylase.

7. The method of claim 6, wherein the composition comprises from about 100 to about 5,000 SKB units of amylase.

8. The method of claim 1, wherein the composition comprises at least three non-pathogenic bacterial strains selected from: Lactobacillus rhamnosus, Bifidobacterium breve, Lactobacillus acidophilus, Bifidobacterium bifidum, Lactobacillus reuteri and combinations thereof.

9. (canceled)

10. The method of claim 1, wherein the non-pathogenic bacterial strain comprises: Lactobacillus rhamnosus, Bifidobacterium breve, and Lactobacillus acidophilus.

11. (canceled)

12. (canceled)

13. The method of claim 1, wherein the non-pathogenic fungal strain is Saccharomyces boulardii.

14. The method of claim 1, wherein the composition is consumed or administered in an amount of comprises 50 mg to 1,000 mg.

15. The method of claim 14, wherein the composition is consumed or administered in an amount of 500 mg to 700 mg.

16. The method of claim 1, wherein the composition comprises from 10 billion to 40 billion colony forming units of the non-pathogenic bacterial strain(s).

17. The method of claim 1, wherein the composition comprises 1 billion to 10 billion of the non-pathogenic fungal strain(s).

18. The method of claim 1, wherein the composition comprises Bifidobacterium breve, Lactobacillus rhamnosus, Lactobacillus acidophilus, Saccharomyces boulardii, and Amylase.

19. The method of any one of claim 1, wherein the alleviation of anxiety disorder or one or more symptoms thereof is measured by reduction in Generalized Anxiety Disorder 7-Item (GAD-7) Scale.

20. A method of alleviating an anxiety disorder or one or more symptoms thereof in a subject in need thereof, the method comprising:
consuming or administering to, the subject a composition comprising (i) an isolated and viable non-pathogenic fungal strain and three different isolated and viable non-pathogenic bacterial strains and (ii) an amylase enzyme capable of disrupting a biofilm and/or breaking down fiber in the subject,
wherein the isolated and viable non-pathogenic fungal strain is Saccharomyces boulardii and the three isolated and viable non-pathogenic bacterial strains are Lactobacillus rhamnosus, Bifidobacterium breve, and Lactobacillus acidophilus,
thereby alleviating the anxiety disorder or one or more symptoms thereof in the subject;
wherein the alleviation of anxiety disorder or one or more symptoms thereof is measured by reduction in Generalized Anxiety Disorder 7-Item (GAD-7) Scale.

21. The method of claim 20, wherein the amylase is selected from the group consisting of Bacillus stearothermophilus amylase, Bacillus amyloliquefaciens amylase, Bacillus subtilis amylase, Bacillus licheniformi amylase, Aspergillus niger amylase, and Aspergillus oryzae amylase.

22. The method of claim 20, wherein the subject is a human.

23. A composition comprising (i) an isolated and viable non-pathogenic fungal strain and three different isolated and viable non-pathogenic bacterial strains and (ii) an amylase enzyme capable of disrupting a biofilm and/or breaking down fiber in the subject for use in a method of alleviating an anxiety disorder or one or more symptoms thereof in a subject in need thereof, the method comprising consumption by, or administration to, the subject of said composition,
wherein the isolated and viable non-pathogenic fungal strain is Saccharomyces boulardii and the three isolated and viable non-pathogenic bacterial strains are Lactobacillus rhamnosus, Bifidobacterium breve, and Lactobacillus acidophilus,
the composition being administered in an effective amount to alleviate the anxiety disorder or one or more symptoms thereof in the subject.


SEQUENCE LISTING (4 sequences)

SEQ ID NO: 1 - DNA, 24 bp, synthetic construct
  misc_feature 1..24: note = Synthetic: 16S-515
  source 1..24: mol_type = other DNA
ggactaccagggtatctaatcctg

SEQ ID NO: 2 - DNA, 19 bp, synthetic construct
  misc_feature 1..19: note = Synthetic: 16S -804
  source 1..19: mol_type = other DNA
tcctacgggaggcagcagt

SEQ ID NO: 3 - DNA, 19 bp, synthetic construct
  misc_feature 1..19: note = Synthetic: fungal ITS1
  source 1..19: mol_type = other DNA
tccgtaggtgaacctgcgg

SEQ ID NO: 4 - DNA, 20 bp, synthetic construct
  misc_feature 1..20: note = Synthetic: ITS4
  source 1..20: mol_type = other DNA
tcctccgcttattgatatgc